(19) United States
(12) Patent Application Publication
(10) Pub. No.: US 2024/0112759 A1
(43) Pub. Date: Apr. 4, 2024

(54) EXPERIMENT ARCHITECT

(71) Applicant: Scinapsis Analytics Inc., dba BenchSci, Toronto (CA)

(72) Inventors: Tom LEUNG, North York (CA); Casandra Savitri MANGROO, Pickering (CA); Luigi GENTILE, Mississauga (CA)

(73) Assignee: Scinapsis Analytics Inc., dba BenchSci, Toronto (CA)

(21) Appl. No.: 17/958,127

(22) Filed: Sep. 30, 2022

(51) Int. Cl. G16B 50/30; G16B 45/00; G16B 50/10

(52) U.S. Cl. CPC G16B 50/30; G16B 45/00; G16B 50/10

(57) ABSTRACT

A method implements an experiment architect. The method includes receiving an entity identifier; searching a plurality of result graphs using the entity identifier to identify a file comprising content corresponding to the entity identifier; and presenting a figure from the file in response to identifying the file.

### BACKGROUND

Biomedical information includes literature and writings that describe evidence from experiments and research of biomedical science that provides the basis for modern medical treatments. Biomedical information is published in publications in physical or electronic form and may be distributed in electronic form using files. Databases of biomedical information provide access to the electronic forms of the publications. A challenge is for computing systems to generate electronic records of experiments and provide relevant evidence related to experiments.

### SUMMARY

In general, in one or more aspects, the disclosure relates to a method of an experiment architect. The method includes receiving an entity identifier; searching a plurality of result graphs using the entity identifier to identify a file comprising content corresponding to the entity identifier; and presenting a figure from the file in response to identifying the file.

In general, in one or more aspects, the disclosure relates to an experiment architect system. The system includes an information controller configured to receive an entity identifier; a search controller configured to search a plurality of result graphs; and an application executing on one or more processors. The application is configured for receiving, by the information controller, the entity identifier; searching, by the search controller, the plurality of result graphs using the entity identifier to identify a file comprising content corresponding to the entity identifier; and presenting a figure from the file in response to identifying the file.

In general, in one or more aspects, the disclosure relates to a non-transitory computer-readable medium storing program instructions that, when executed by one or more processors, cause a computing system to perform operations of an experiment architect system. The instructions are configured for receiving an entity identifier; searching a plurality of result graphs using the entity identifier to identify a file comprising content corresponding to the entity identifier; and presenting a figure from the file in response to identifying the file.

Other aspects of the invention will be apparent from the following description and the appended claims.

### BRIEF DESCRIPTION OF DRAWINGS

FIG. 1A, FIG. 1B, FIG. 1C, and FIG. 1D show diagrams of systems in accordance with disclosed embodiments.

FIG. 2 shows a flowchart in accordance with disclosed embodiments.

FIG. 3A, FIG. 3B, FIG. 4, FIG. 5, FIG. 6, FIG. 7A, FIG. 7B, FIG. 7C, FIG. 8A, FIG. 8B, FIG. 8C, FIG. 8D, FIG. 8E, FIG. 9A, FIG. 9B, FIG. 9C, FIG. 10A, FIG. 10B, FIG. 10C, FIG. 10D, FIG. 11, and FIG. 12 show examples in accordance with disclosed embodiments.

FIG. 13A and FIG. 13B show computing systems in accordance with disclosed embodiments.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure architect experiments. Users interact with the system to provide information describing experiments. The system searches evidence result graphs generated from biomedical information to the information provided by users to provide biomedical information relevant to experiments of the users.

In general, embodiments of the disclosure implement evidence result networks. An evidence result network may be implemented as a graph generated from a file or other scientific evidence by a computing system. The system receives a file (i.e., including but not limited to publications of biomedical information, patents, documents of experimental results originating from external and internal customer sources, etc.) that stores the evidence using text, images, data, etc. The evidence (i.e., the data of a file describing scientific results) is processed to identify the sentences and images in the file. The file may be referred to as a source of evidence of a scientific result. One or more machine learning models may be used to process the sentences and images to generate graphs (evidence result networks) that represent the evidence demonstrated from experiments described by the sentences and images from the file. An ontology library (saved as a collection of data records) is used to identify terms and phrases from the text and images of the file that relate to entities with biomedical meaning. For example, the ontology library may store the names of proteins, diseases, experimentation techniques, etc. The entities from the ontology library may be recognized during the processing of the file to preserve the meaning of terms and phrases from the text and images in the graphs generated by the system.

The machine learning models used by the system may be trained to understand evidence both written and visual. For example, a machine learning model may be trained to recognize and tag entities in biomedical information, defined by the data records of the ontology library, that appear in a sentence. Additional machine learning models (semantic tree generators, image recognizers, etc.) may be trained with biomedical data (text and images) to be customized for biomedical data.

After a file is processed to generate a set of result graphs for the evidence described by the data of the file, the graphs and images from the file may be displayed to a user. For example, a user interested in the relationship between two entities (e.g., a protein and a disease) may locate a file corresponding to a biomedical publication that includes the two entities in a graph generated from a sentence or image from the file. Graphs and images that describe the relationships between the entities may then be displayed to the user.

The figures show diagrams of embodiments that are in accordance with the disclosure. The embodiments of the figures may be combined and may include or be included within the features and embodiments described in the other figures of the application. The features and elements of the figures are, individually and as a combination, improvements to the technology of biomedical information processing and machine learning models. The various elements, systems, components, and steps shown in the figures may be omitted, repeated, combined, and/or altered as shown from the figures. Accordingly, the scope of the present disclosure should not be considered limited to the specific arrangements shown in the figures.

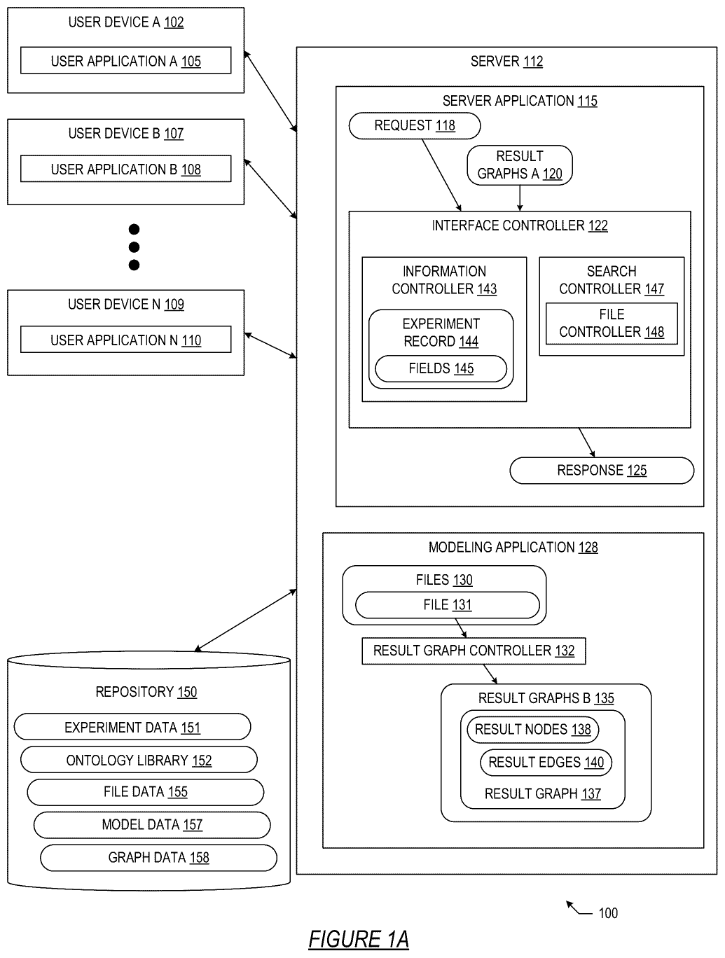

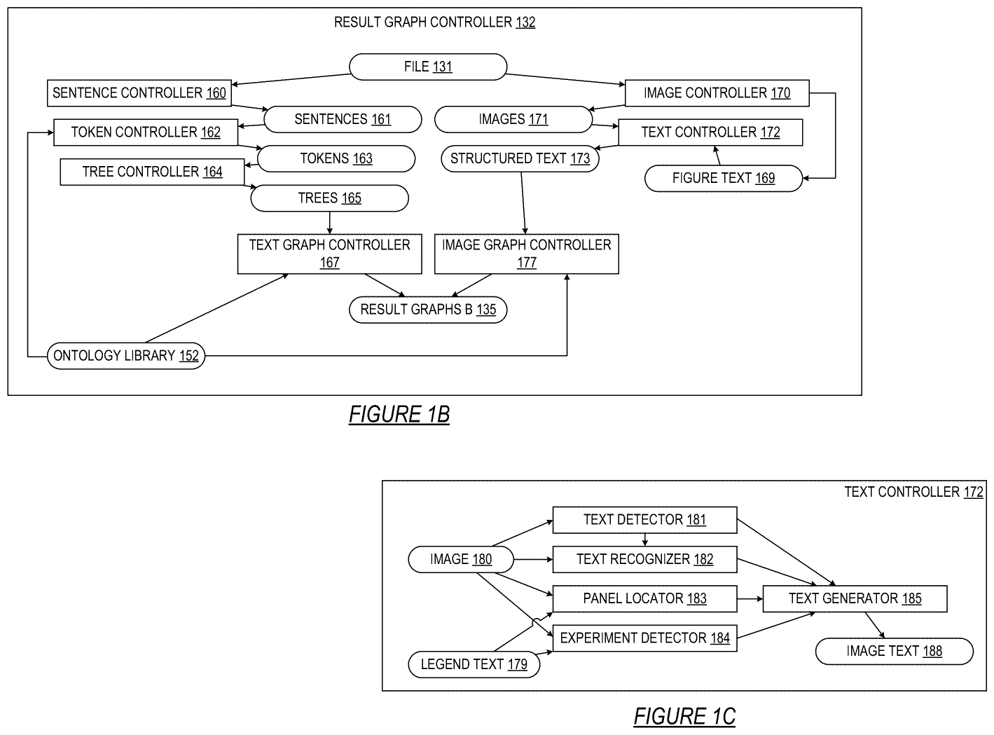

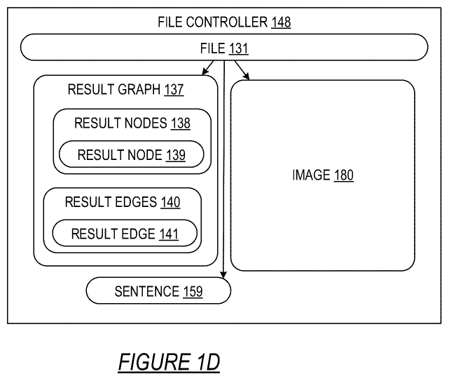

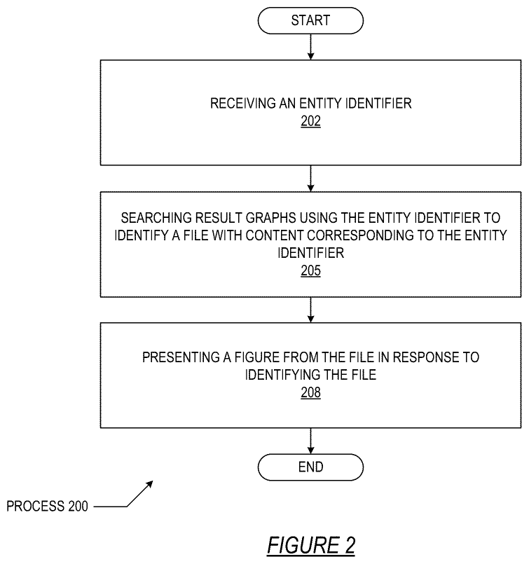

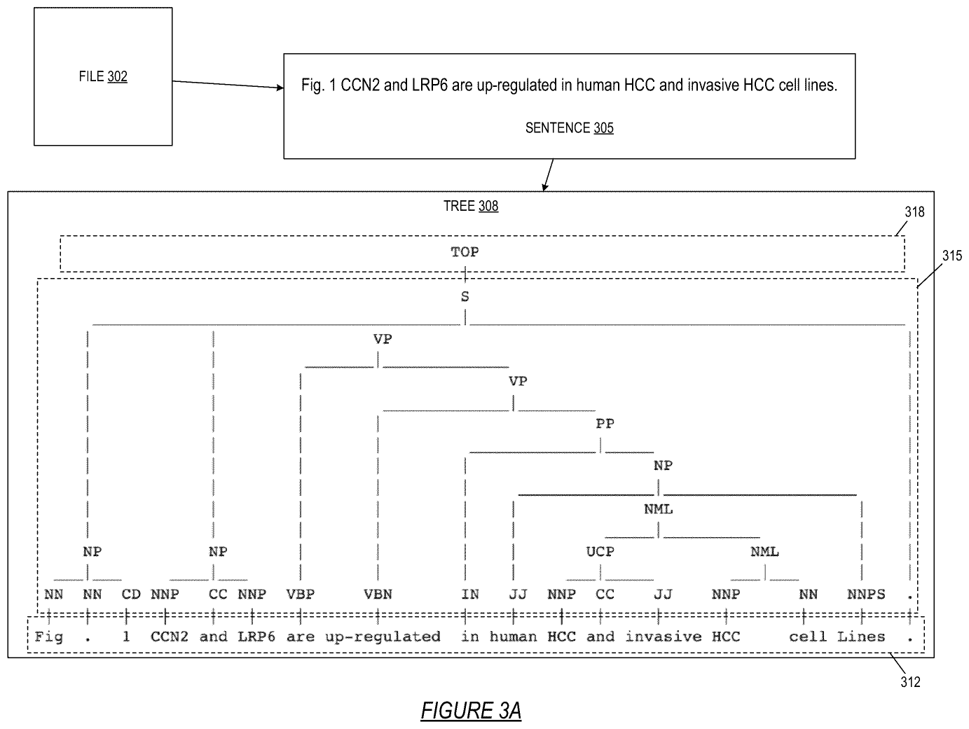

Turning to FIG. 1A, the system (100) architects experiments by interacting with users to record and search for biomedical information. The system (100) receives requests (e.g., the request (118)) and generates responses (e.g., the response (125)) using the result graphs A (120). The system (100) generates the result graphs A (120) from biomedical information (e.g., the files (131)) stored in the file data (155) using multiple machine learning and natural language processing models. The system (100) uses the result graphs A (120) to populate search results with biomedical information relevant to the biomedical information requested by users. The system (100) generates the response (125) using the result graphs A (120). The system (100) may display the result graphs A (120) and the images from the files of the file data (155) to users operating the user devices A (102) and B (107) through N (109). The system (100) includes the user devices A (102) and B (107) through N (109), the server (112), and the repository (150).

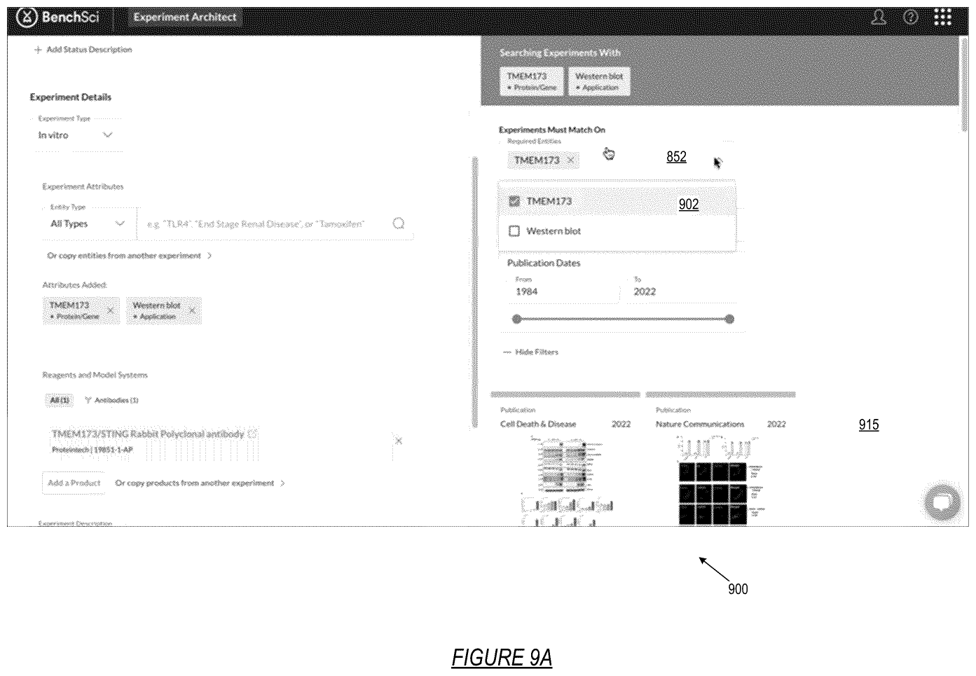

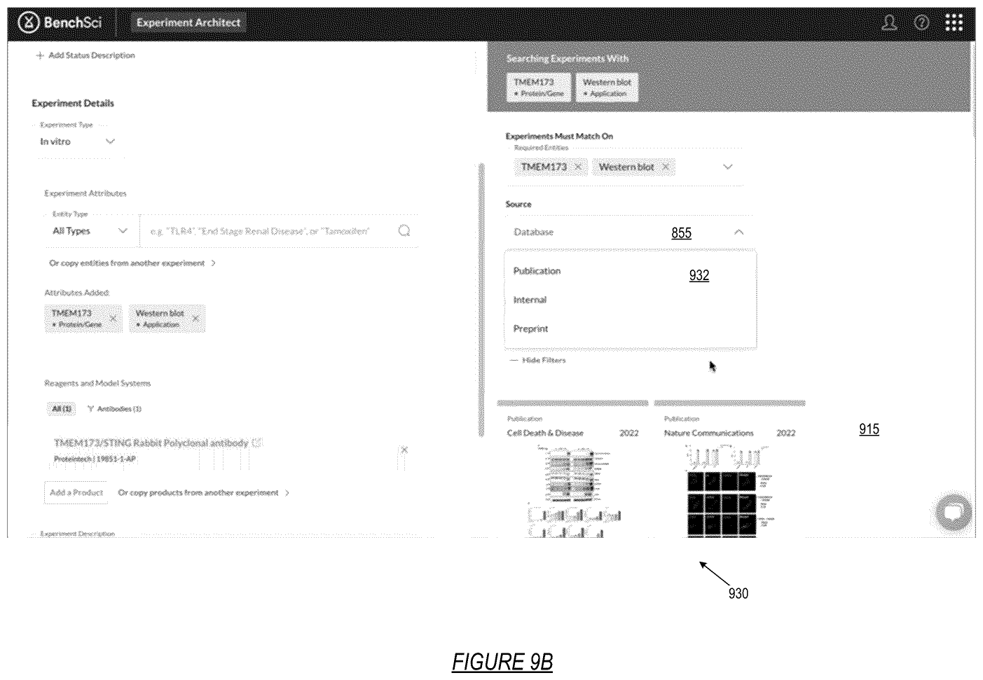

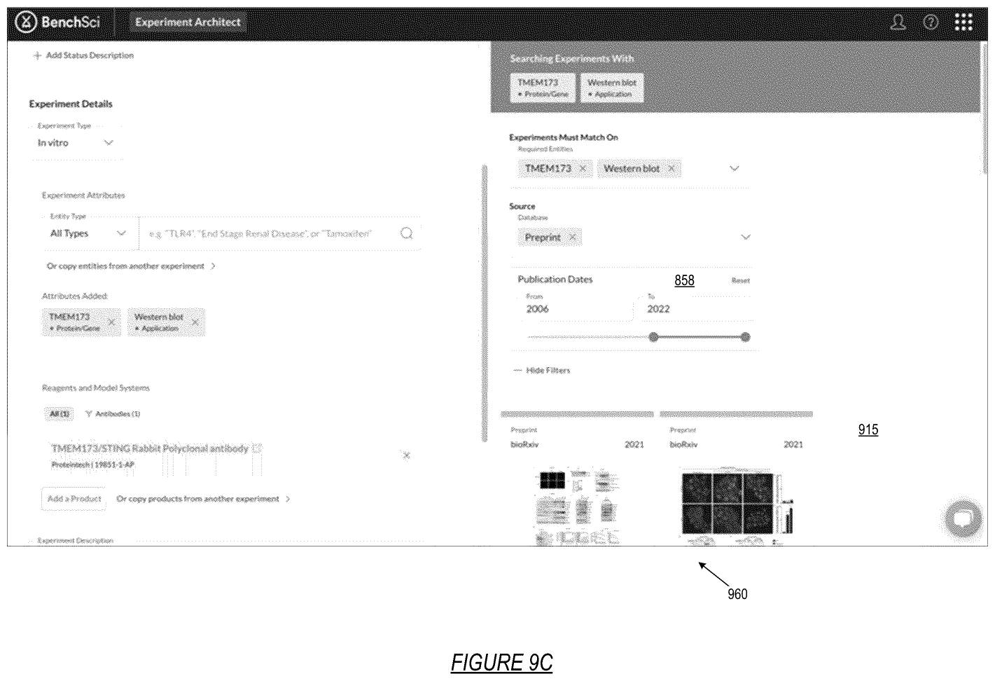

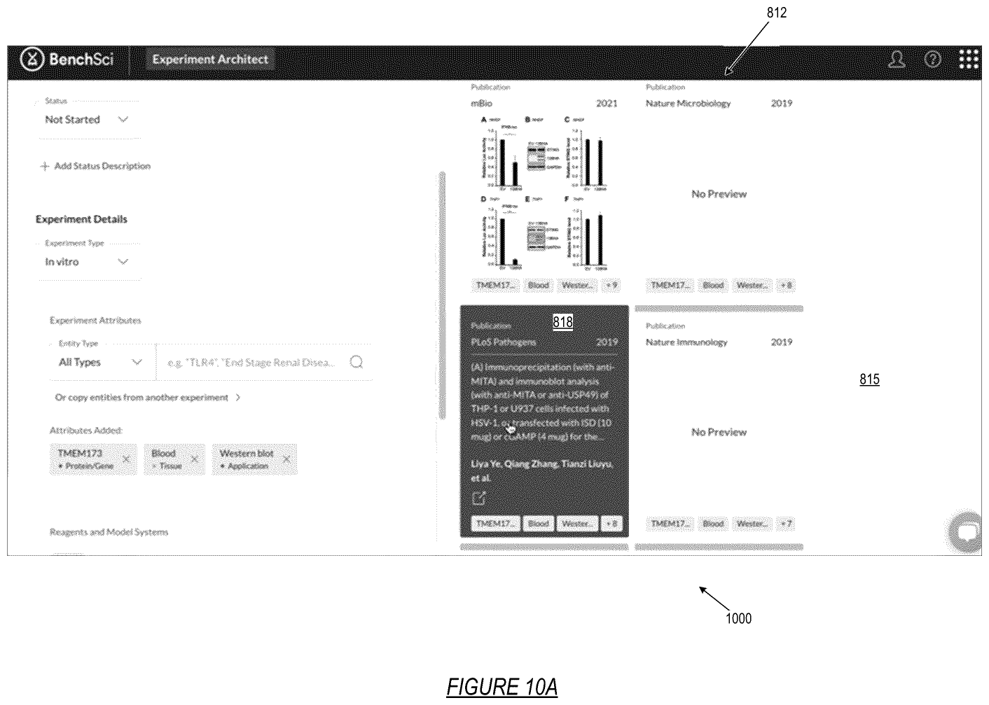

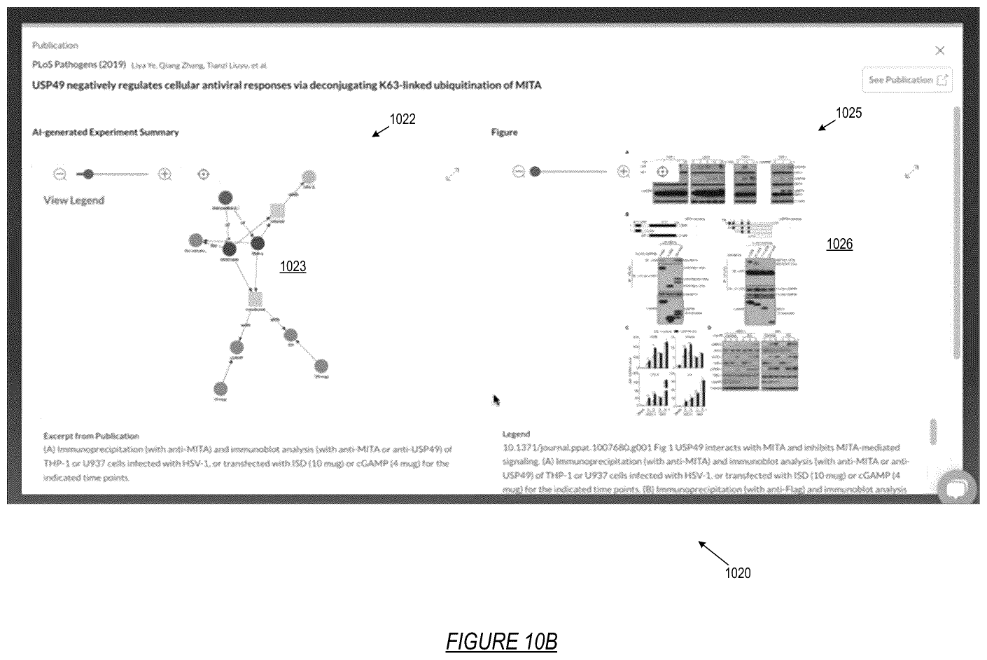

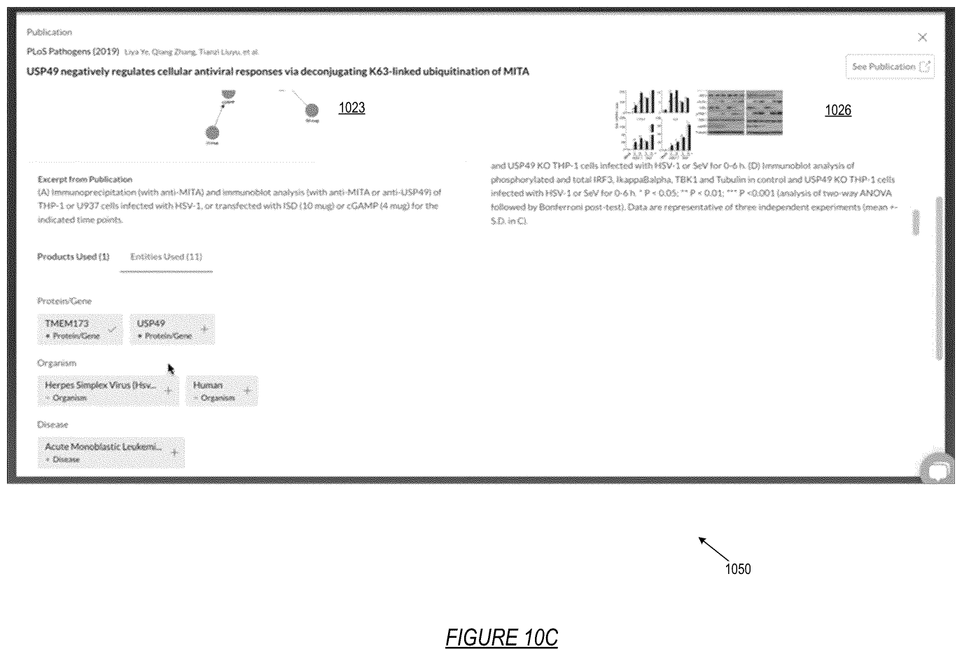

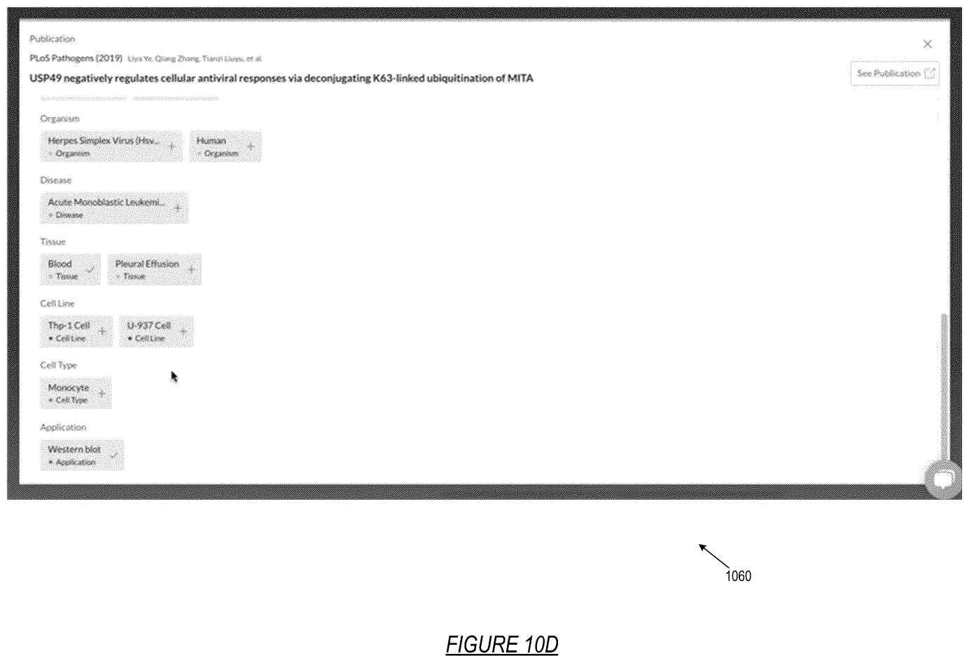

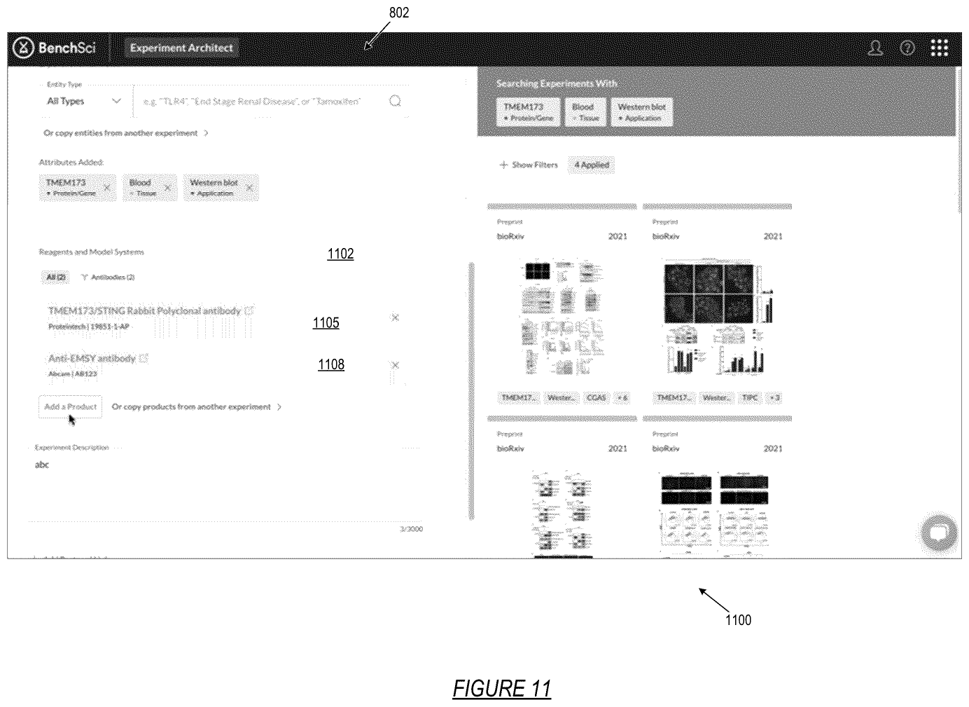

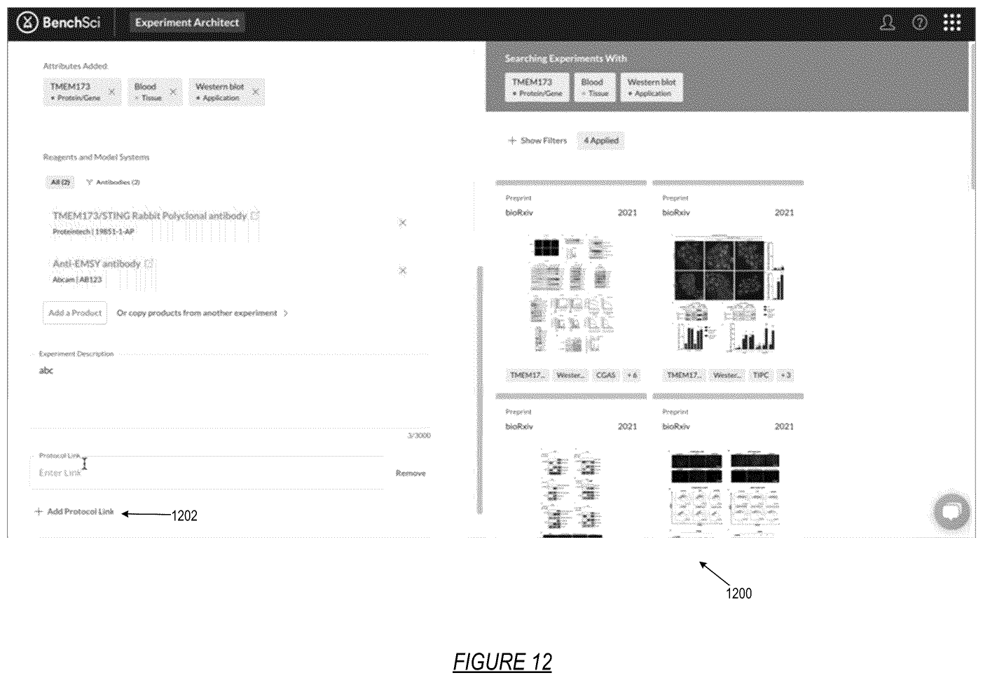

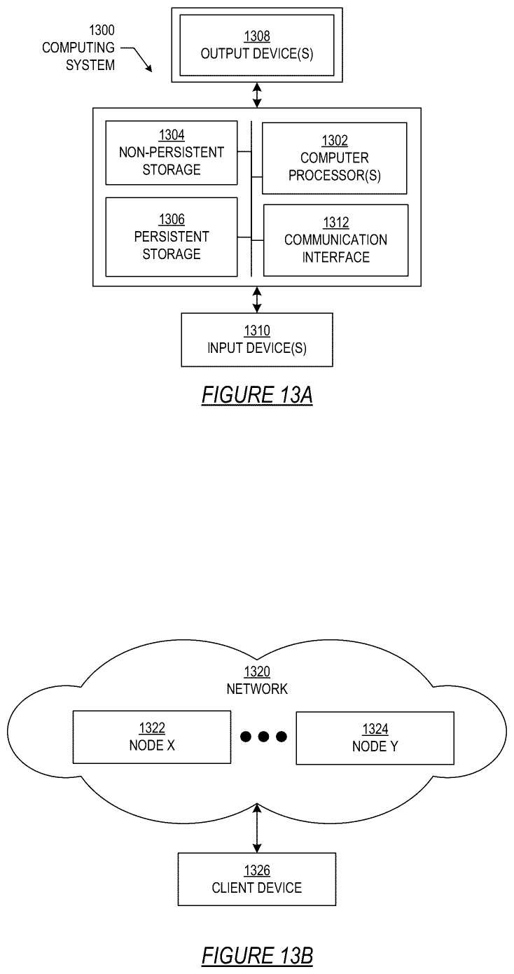

The server (112) is a computing system (further described in FIG. 13A). The server (112) may include multiple physical and virtual computing systems that form part of a cloud computing environment. In one embodiment, execution of the programs and applications of the server (112) is distributed to multiple physical and virtual computing systems in the cloud computing environment. The server (112) includes the server application (115) and the modeling application (128).

The server application (115) is a collection of programs that may execute on multiple servers of a cloud environment, including the server (112). The server application (115) receives the request (118) and generates the response (125) based on the result graphs A (120) using the interface controller (122). The server application (115) may host websites accessed by users of the user devices A (102) and B (107) through N (109) to view and interact with information using the information controller (143) and the search controller (147). The websites hosted by the server application (115) may serve structured documents (hypertext markup language (HTML) pages, extensible markup language (XML) pages, JavaScript object notation (JSON) files and messages, etc.). The server application (115) includes the interface controller (122), which processes the request (118) using the result graphs A (120).

The request (118) is a request from one of the user devices A (102) and B (107) through N (109). In one embodiment, the request (118) is a request for recording biomedical information of an experiment being planned by a user. The biomedical information may include one or more entities defined in the ontology library (152), described in the file data (155), and graphed in the graph data (158).

The result graphs A (120) are generated with the modeling application (128), described further below. The result graphs A (120) includes nodes and edges in which the nodes correspond to text from the file data (155) and the edges correspond to semantic relationships between the nodes. The result graphs A (120) are directed graphs in which the edges identify a direction from one node to a subsequent node in the result graphs A (120). In one embodiment, the result graphs A (120) are acyclic graphs. The result graphs A (120) may be stored in the graph data (158) of the repository (150).

The interface controller (122) is a collection of programs that may operate on the server (112). The interface controller (122) processes the request (118) using the result graphs A (120) to generate the response (125). In one embodiment, the interface controller (122) searches the graph data (158) to identify the result graphs A (120) (which may include some of the result graphs from the result graphs B (135)) that include information about the entities identified in the request (118). The interface controller (122) uses the information controller (143) and the search controller (147) to process the request (118).

The information controller (143) is a collection of programs that may operate on the server (112). The information controller (143) presents and stores biomedical information of an experiment. The biomedical information is recorded in the experiment record (144).

The experiment record (144) stores biomedical information of an experiment. The experiment record (144) is one of multiple experiment records stored in the experiment data (151) of the repository (150). The experiment record (144) includes multiple fields.

The fields (145) store the data of the experiment record (144). The fields (145) store biomedical information describing the experiment corresponding to the experiment record (144). The fields (145) may store numeric information, categorical information, text information, etc. In one embodiment, the fields (145) include fields for a name, an objective, a status, details, attributes, reagents and model systems, a description, a protocol, etc. The fields (145) are stored in the experiment data (151) of the repository (150).

The search controller (147) is a collection of programs that may operate on the server (112). The search controller (147) searches the file data (155) using the graph data (158). For example, the search controller (147) may search the result graphs B (135) for the entities identified in the attributes from the fields (145) of the experiment record (144) to identify the result graphs A (120). The search controller (147) may display results with the file controller (148).

The file controller (148) is a collection of programs that may operate on the server (112). In one embodiment, the file controller (148) may present biomedical information from a file (sentence, result graph, image, etc.) from the file data (155).

The response (125) is generated by the interface controller (122) in response to the request (118) using the result graphs A (120). In one embodiment, the response (125) includes images from the file data (155) corresponding to the result graphs A (120). Portions of the response (125) may be displayed by the user devices A (102) and B (107) through N (109) that receive the response (125).

The modeling application (128) is a collection of programs that may operate on the server (112). The modeling application (128) generates the result graphs B (135) from the files (130) using a result graph controller (132).

The files (130) include biomedical information and form the basis for the result graphs B (135). The files (130) include the file (131), which is the basis for the result graph (137). As an example, the file (131) may be a publication of biomedical information. Each file includes multiple sentences and may include multiple images of evidence. The evidence may identify how different entities, defined in the ontology library (152), affect each other. For example, entities that are proteins may suppress or enhance the expression of other entities and affect the prevalence of certain diseases. Types of entities include proteins, genes, diseases, experiment techniques, chemicals, cell lines, pathways, tissues, cell types, organisms, etc. In one embodiment, nouns and verbs from the sentences of the file (131) are mapped to the result nodes (138) of the result graph (137). In one embodiment, the semantic relationships between the words in the sentences corresponding to the result nodes (138) are mapped to the result edges (140). In one embodiment, one file serves as the basis for multiple result graphs. In one embodiment, one sentence from a file may serve as the basis for one result graph.

The result graph controller (132) generates the result graphs B (135) from the files (130). The result graph controller (132) is a collection of programs that may operate on the server (112). For a sentence of the file (131), the result graph controller (132) identifies the result nodes (138) and the result edges (140) for the result graph (137).

The result graphs B (135) are generated from the files (130) and includes the result graph (137), which corresponds to the file (131). The result nodes (138) represent nouns and verbs from a sentence of the file (131). The result edges (140) identify semantic relationships between the words represented by the result nodes (138).

The user devices A (102) and B (107) through N (109) are computing systems (further described in FIG. 13A). For example, the user devices A (102) and B (107) through N (109) may be desktop computers, mobile devices, laptop computers, tablet computers, server computers, etc. The user devices A (102) and B (107) through N (109) include hardware components and software components that operate as part of the system (100). The user devices A (102) and B (107) through N (109) communicate with the server (112) to access, manipulate, and view information, including information from the graph data (158) and the file data (155). In one embodiment, the user devices A (102) and B (107) through N (109) may communicate with the server (112) using standard protocols and file types, which may include hypertext transfer protocol (HTTP), HTTP secure (HTTPS), transmission control protocol (TCP), internet protocol (IP), hypertext markup language (HTML), extensible markup language (XML), etc. The user devices A (102) and B (107) through N (109) respectively include the user applications A (105) and B (108) through N (110).

The user applications A (105) and B (108) through N (110) may each include multiple programs respectively running on the user devices A (102) and B (107) through N (109). The user applications A (105) and B (108) through N (110) may be native applications, web applications, embedded applications, etc. In one embodiment, the user applications A (105) and B (108) through N (110) include web browser programs that display web pages from the server (112). In one embodiment, the user applications A (105) and B (108) through N (110) provide graphical user interfaces that display information stored in the repository (150).

As an example, the user application A (105) may be operated by a user and generate the request (118) to view and generate the experiment record (144) and view information related to entities defined in the ontology library (152), described in the file data (155), and graphed in the graph data (158). Corresponding sentences and images from the file data (155) and graphs from the graph data (158) may be received in the response (125) and displayed in a user interface of the user application A (105).

As another example, the user device N (109) may be used by a developer to maintain the software applications hosted by the server (112) and train the machine learning models used by the system (100). Developers may view the data in the repository (150) to correct errors or modify the application served to the users of the system (100).

The repository (150) is a computing system that may include multiple computing devices in accordance with the computing system (1300) and the nodes (1322) and (1324) described below in FIGS. 13A and 13B. The repository (150) may be hosted by a cloud services provider that also hosts the server (112). The cloud services provider may provide hosting, virtualization, and data storage services as well as other cloud services and to operate and control the data, programs, and applications that store and retrieve data from the repository (150). The data in the repository (150) includes the experiment data (151), the ontology library (152), the file data (155), the model data (157), and the graph data (158).

The experiment data (151) includes information describing experiments generated by the users of the system (100). In one embodiment, the experiment data (151) stores information as data in fields of experiment records (e.g., the fields (145) of the experiment record (144)). The fields store biomedical information describing experiments and may include numeric information, categorical information, text information, etc. In one embodiment, the experiment data (151) includes experiment records with fields for a name, an objective, a status, details, attributes, reagents and model systems, a description, a protocol, etc. The fields (145) are stored in the experiment data (151) of the repository (150).

The name identifies the experiment. The name may be stored as a string.

The objective describes the purpose of the experiment. The objective may be stored as text.

The status identifies the completion status of the experiment. In one embodiment, the status is stored as a numeric data type that identifies one of a finite set of categories for the completion status, which may include “0—Not Started”, “1—Started”, “2—Completed”, etc. In one embodiment, the status may be stored as a text string that describes the completion status.

The details describe details of the experiment. The details may be stored as a text string that may include multiple paragraphs of text.

The attributes identify entities from the ontology library that are related to the experiment. The system (100) searches the result graphs B (135) based on the attributes to identify the result graphs A (120), which include biomedical information pertinent to the experiment of the experiment record (144). The attributes may be stored as a list of codes, tags, strings, etc.

The reagents and model systems are a list of products that identify the reagents and models systems to be used in the experiment. The products that may be identified by the vendor and shelf keeping unit (SKU) codes. Multiple products may used for one experiment and may be identified using one or more text strings. For example, a vendor string and an SKU string may be used to identify a product.

The description describes the experiment. The description may be stored as a string with multiple sentences and paragraphs.

The protocol describes the steps used to perform the experiment. In one embodiment, the protocol may include a link to another web page or file that describes the protocol used for the experiment.

The ontology library (152) includes information that of the entities and biomedical terms and phrases used by the system (100). Multiple terms and phrases may be used for the same entity. The ontology library (152) defines types of entities. In one embodiment, the types include the types of protein/gene, chemical, cell line, pathway, tissue, cell type, disease, organism, etc. The ontology library (152) may store the information about the entities in a database, structured text files, combinations thereof, etc.

The file data (155) is biomedical information stored in electronic records. The biomedical information describes the entities and corresponding relationships that are defined and stored in the ontology library (152). The file data (155) includes the files (130). Each file in the file data (155) may include image data and text data. The image data includes images that represent the graphical figures from the files. The text data represents the writings in the file data (155). The text data for a file includes multiple sentences that each may include multiple words that each may include multiple characters stored as strings in the repository (150). In one embodiment, the file data (155) includes biomedical information stored as extensible markup language (XML) files, portable document files (PDFs). The file formats define containers for the text and images of the biomedical information describing evidence of biomedical experiments.

The model data (157) includes the data for the models used by the system (100). The models may include rules-based models and machine learning models. The machine learning models may be updated by training, which may be supervised training. The modeling application (128) may load the models from the model data (157) to generate the result graphs B (135) from the files (130).

The model data (157) may also include intermediate data. The intermediate data is data generated by the models during the process of generating the result graphs B (135) from the files (130).

The graph data (158) is the data of the graphs (including the result graphs A (120) and B (135)) generated by the system. The graph data (158) includes the nodes and edges for the graphs. The graph data (158) may be stored in a database, structured text files, combinations thereof, etc.

Although shown using distributed computing architectures and systems, other architectures and systems may be used. In one embodiment, the server application (115) may be part of a monolithic application that manipulate biomedical information. In one embodiment, the user applications A (105) and B (108) through N (110) may be part of monolithic applications that manipulate biomedical information without the server application (115).

Turning to FIG. 1B, the result graph controller (132) processes the file (131) to generate the result graphs B (135). In one embodiment, the result graph controller (132) includes the sentence controller (160), the token controller (162), the tree controller (164), and the text graph controller (167) to process the text from the file (131) describing biomedical evidence. In one embodiment, the result graph controller (132) includes the image controller (170), the text controller (172), and the image graph controller (177) to process the figures from the file (131) that provide evidence for the conclusions of experiments.

The sentence controller (160) is a set of programs that operate to extract the sentences (161) from the file (131). In one embodiment, the sentence controller (160) cleans the text of the file (131) by removing markup language tags, adjusting capitalization, etc. The sentence controller (160) may split a string of text into substrings with each substring being a string that includes a sentence from the original text of the file (131). In one embodiment, the sentence controller (160) may filter the sentences and keep sentences with references to the figures of the file (131).

The sentences (161) are text strings extracted from the file (131). A sentence of the sentences (161) may be stored as a string of text characters. In one embodiment, the sentences (161) are stored in a list that maintains the order of the sentences (161) from the file (131). In one embodiment, the list may be filtered to remove sentences that do not contain a reference to a figure.

The token controller (162) is a set of programs that operate to locate the tokens (163) in the sentences (161). The token controller (162) may identify the start and stop of each token in a sentence

The tokens (163) identify the boundaries of words in the sentences (161). In one embodiment, a token (of the tokens (163)) may be a substring of a sentence (of the sentences (161)). In one embodiment, a token (of the tokens (163)) may be a set of identifiers that identify the locations of a start character and a stop character in a sentence. Each sentence may include multiple tokens.

The tree controller (164) is a set of programs that operate to generate the trees (165) from the tokens (163) of the sentences (161) of the file (131). In one embodiment, the tree controller (164) uses a neural network (e.g., the Berkeley Neural Parser)

The trees (165) are syntax trees of the sentences (161) to identify the parts of speech of the tokens (163) within the sentences (161). In one embodiment, the trees (165) are graphs with edges identifying parent child relationships between the nodes of a graph. In one embodiment, the nodes of a graph of a tree include a root node, intermediate nodes, and leaf nodes. The leaf nodes correspond to tokens (words, terms, multiword terms, etc.) from a sentence and the intermediate nodes identify parts of speech of the leaf nodes.

The text graph controller (167) is a set of programs that operate to generate the result graphs B (135) from the trees (165). In one embodiment, the text graph controller (167) maps the tokens (163) from the sentences (161) that represent nouns and verbs to nodes of the result graphs B (135). In one embodiment, the text graph controller (167) maps parts of speech identified by the trees (165) to the edges of the result graphs B (135).

In one embodiment, after generating an initial graph (of the result graphs B (135)) for a sentence (of the sentences (161)), the text graph controller (167) processes the graph using the ontology library (152) to identify the entities and corresponding entity types represented by the nodes of the graph. For example, a node of the graph may correspond to the token “BRD9”. The text graph controller (167) identifies the token as an entity defined in the ontology library (152) and identifies the entity type as a protein.

The image controller (170) is a set of programs that operate to extract figures from the file (131) to generate the images (171). The image controller also extracts the figure text (169) that corresponds to the images (171). In one embodiment, the image controller (170) may use rules and logic to identify the images and corresponding image text from the file (131). In one embodiment, the image controller (170) may use machine learning models to identify the images (171) and the figure text (169). For example, the file (131) may be stored in a page friendly format (e.g., a portable document file (PDF)) in which each page of a publication is stored as an image in a file. A machine learning model may identify pages that include figures and the locations of the figures on those pages. The located figures may be extracted as the images (171). Another machine learning model may identify the legend text that corresponds to and describes the figures, which is extracted as the figure text (169).

The images (171) are image files extracted from the file (131). In one embodiment, the file (131) includes the figures as individual image files that the image controller (170) converts to the images (171). In one embodiment, the figures of the file (131) may be contained within larger images, e.g., the image of a page of the file (131). The image controller (170) processes the larger images to extract the figures as the images (171).

The figure text (169) is the text from the file (131) that describes the images (171). Each figure of the file (131) may include legend text that describes the figure. The legend text for one or more figures of the file (131) is extracted as the figure text (169), which corresponds to the images (171).

The text controller (172) is a set of programs that operate to process the images (171) and the figure text (169) to generate the structured text (173). The text controller (172) is further described with FIG. 1C below.

The structured text (173) is strings of nested text with information extracted from the images (171) using the figure text (169). In one embodiment, the structured text (173) includes a JSON formatted string for each image of the images (171). In one embodiment, the structured text (173) identifies the locations of text, panels, and experiment metadata within the images (171). In one embodiment, the structured text (173) includes text that is recognized from the images (171). The structured text (173) may include additional metadata about the images (171). For example, the structured text may identify the types of experiments and the types of techniques used in the experiments that are depicted in the images (171).

The image graph controller (177) is a set of programs that operate to process the structured text (173) to generate one or more of the result graphs B (135). In one embodiment, the image graph controller (177) identifies text that corresponds to entities defined in the ontology library (152) from the structured text (173) and maps the identified text to nodes of the result graphs B (135). In one embodiment, the image graph controller (177) uses the nested structure of the structure text (173) to identify the relationships between the nodes of one or more of the result graphs B (135) and maps the relationships to edges of one or more of the result graphs B (135).

The result graphs B (135) are the graphs generated from the file (131) by the result graph controller (132). The result graphs B (135) include nodes that represent entities defined in the ontology library (152) and include edges that represent relationships between the nodes.

The ontology library (152) defines the entities that may be recognized by the result graph controller (132) from the file (131). The entities defined by the ontology library (152) are input to the token controller (162), the text graph controller (167), and the image graph controller (177), which identify the entities within the text and image extracted from the file (131).

Turning to FIG. 1C, the text controller (172) processes the image (180) and the corresponding legend text (179) to generate the image text (188). The text controller (172) may operate as part of the result graph controller (132) of FIG. 1B.

The image (180) is one of the images (171) from FIG. 1B. The image (180) includes a figure from the file (131) of FIG. 1B.

The legend text (179) is a string from the figure text (169) of FIG. 1B. The legend text (179) is the text from the legend of the figure that corresponds to the image (180).

The text detector (181) is a set of programs that operate to process the image (180) to identify the presence and location of text within the image (180). In one embodiment, the text detector (181) uses machine learning models to identify the presence and location of text. The location may be identified with a bounding box that specifies four points of a rectangle that surrounds text that has been identified in the image (180). The location of the text from the text detector (181) may be input to the text recognizer (182).

The text recognizer (182) is a set of programs that operates to process the image (180) to recognize text within the image (180) and output the text as a string. The text recognizer (182) may process a sub image from the image (180) that corresponds to a bounding box identified by the text detector (181). A machine learning model may then be used to recognize the text from the sub image and output a string of characters that correspond to the text within the sub image.

The panel locator (183) is a set of programs that operates to process the image (180) to identify the location of panels and subpanels within the image (180) or a portion of the image (180). A panel of the image (180) is a portion of the image, which may depict evidence of an experiment. The panels of the image (180) may contain subpanels to further subdivide information contained within the image (180). The image (180) may include multiple panels and subpanels that may identified within the legend text (179). The panel locator (183) may be invoked to identify the location for each panel (or subpanel) identified in the legend text (179). In one embodiment, the panel locator (183) outputs a bit array with each bit corresponding to a pixel from the image (180) and identifying whether the pixel corresponds to a panel.

The experiment detector (184); is a set of programs that operates to process the image (180) to identify metadata about experiments depicted in the image (180). In one embodiment, the experiment detector (184) processes the image (180) with a machine learning model (e.g., a convolutional neural network) that outputs a bounding box and a classification. In one embodiment, the bounding box may be an array of coordinates (e.g., top, left, bottom, right) in the image that identify the location of evidence of an experiment within the image. In one embodiment, the classification may be a categorical value that identifies experiment metadata, which may include the type of evidence, the type of experiment, or technique used in the experiment (e.g., graph, western blot, etc.).

The text generator (185) is a set of programs that operate to process the outputs from the text detector (181), the text recognizer (182), the panel locator (183), and the experiment detector (184) to generate the image text (188). In one embodiment, the text generator (185) creates a nested structure for the image text (188) based on the outputs from the panel locator (183), the experiment detector (184), and the text detector (181). For example, the text generator (185) may include descriptions for the panels, experiment metadata, and text from the image (180) in which the text and description of the experiment metadata may be nested within the description of the panels. Elements for subpanels may be nested within the elements for the panels.

The image text (188) is a portion of the structured text (173) of FIG. 1B that corresponds to the image (180). In one embodiment, the image text (188) uses a nested structure to describe the panels, experiment metadata, and text that are identified and located within the image (180).

Turning to FIG. 1D, the file controller (148) presents information from the file (131). In one embodiment, the file controller (148) updates the response (125) (of FIG. 1A) to include the result graph (137), the image (180), and the sentence (159) from the file (131).

The file (131) is a file of biomedical information. The biomedical information from the file (131) may be presented with the search controller (147). The file (131) is stored in the file data (155) (of FIG. 1A), includes the sentence (159) (from which the result graph (137) is generated) and includes the image (180), which corresponds to the sentence (159).

The result graph (137) is a result graph generated from the sentence (159) from the file (131). The result graph includes the result nodes (138) (including the result node (139)) and includes the result edges (140) (including the result edge (141)). The result node (139) may correspond to the same entity to which the evidence node (124) corresponds. The result edge (141) may identify a semantic relationship between the result node (139) and another result node from the result graph (137).

The sentence (159) is a sentence from the file (131). The sentence (159) is parsed to generate the result graph (137).

The image (180) is an image from the file (131). The image (180) comprises a figure from the file (131) that is referred to in the sentence (159).

Turning to FIG. 2, the process (200) architects experiments. The process (200) may be performed by a computing system, such as the computing system (1300) of FIG. 13A.

At Step 202, an entity identifier is received. In one embodiment, the entity identifier may be received by the user interface of a user device, transmitted to a server, and received by the server. In one embodiment, the entity identifier corresponds to an entity node of the result graph and corresponds to an entity record of an ontology library. In one embodiment, the entity record defines an entity as one of a protein, a gene, a disease, a cell line, a cell type, a tissue, a pathway, an organism, a chemical, and an application.

In one embodiment, the entity identifier is received in response to a user selection of the entity identifier. The user may select the entity identifier by entering text that corresponds to the entity identifier into a user interface displayed on a user device.

In one embodiment, the entity identifier is received in response to processing a user selection to identify the entity identifier. For example, a user may input a name of an experiment, the system processes the name to identify words or phrases from the name that correspond to entities of the ontology library. The system may compare the words from the text entered by the user to the names and aliases of the entities defined in the ontology library.

In one embodiment, the entity identifier is received in response to a user selection of an alias of an entity corresponding to the entity identifier. For example, the system may identify multiple aliases that correspond to text entered by the user and the user may select one of the aliases.

In one embodiment, one or more values of an experiment record are processed to select the entity identifier. For example, the values of the experiment record may include names, aliases, words, and phrases, that are matched to text input by the user. In one embodiment, regular expressions may be used to identify matches between text input by the user and values from entity records. In one embodiment, the entity identifier may be received in response to (i.e., after) processing the one or more values of the experiment record.

At Step 205, result graphs are searched using the entity identifier to identify a file comprising content corresponding to the entity identifier. In one embodiment, the file comprises biomedical information. In one embodiment, the biomedical information comprises text and images.

In one embodiment, the plurality of result graphs are searched to identify an entity node, of the result graph, corresponding to the entity identifier. In one embodiment, the entity identifier corresponds to a name of an entity that is compared to the values corresponding to the entity nodes of the result graphs. When the name of an entity corresponds to both the entity identifier and to an entity node from a result graph, the result graph is identified as a match and may be displayed to the user.

At Step 208, a figure from the file is presented in response to identifying the file. The figure may be presented by transmitting the figure from a server to a user device and then displaying the figure on the user device in a user interface. In one embodiment, one of the result graphs was generated from the file and the file includes the figure.

In one embodiment, an evidence view is presented adjacent to a results view. The evidence view may be part of a user interface that a user interacts with to view and input details of a record of an experiment. The results view may be part of a user interface that a user interacts with to see evidence (e.g., publications) that are related to the experiment record being created by the user.

In one embodiment, the entity identifier is received using the evidence view. After receiving the entity identifier, the figure is presented in the results view. The figure is evidence of biomedical information described in the file and corresponds to the result graph.

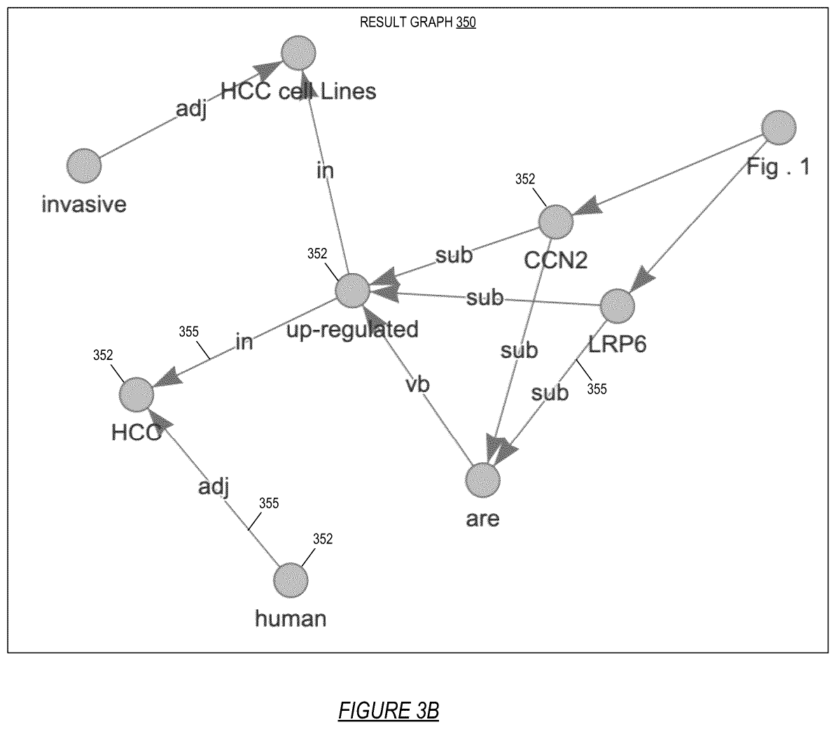

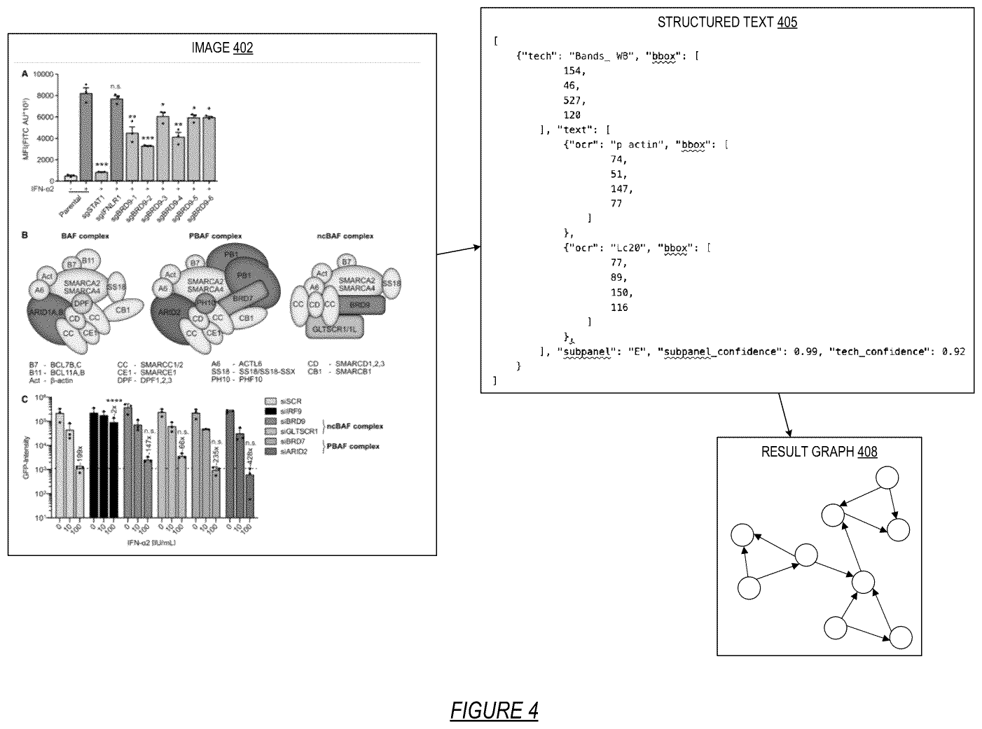

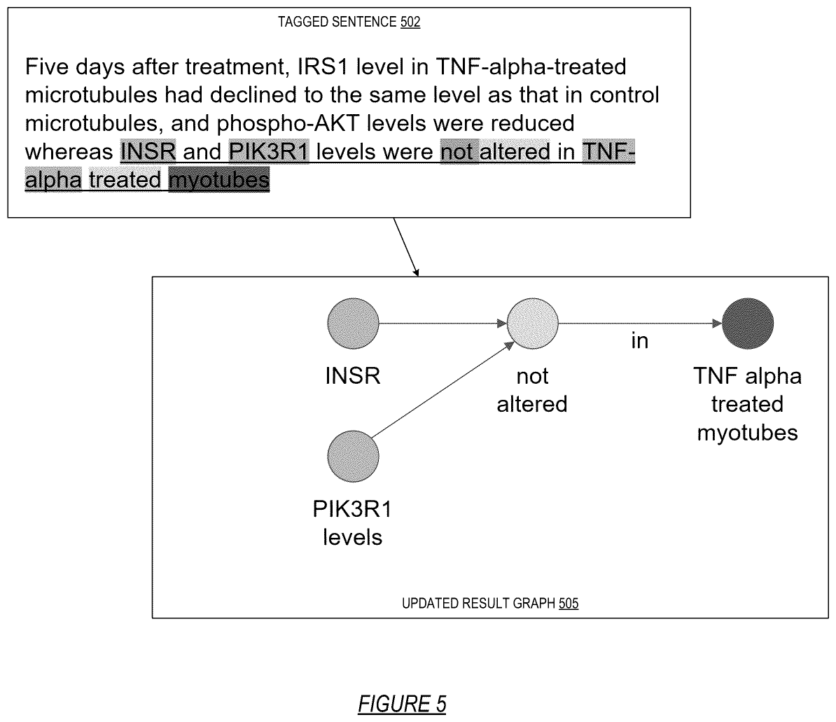

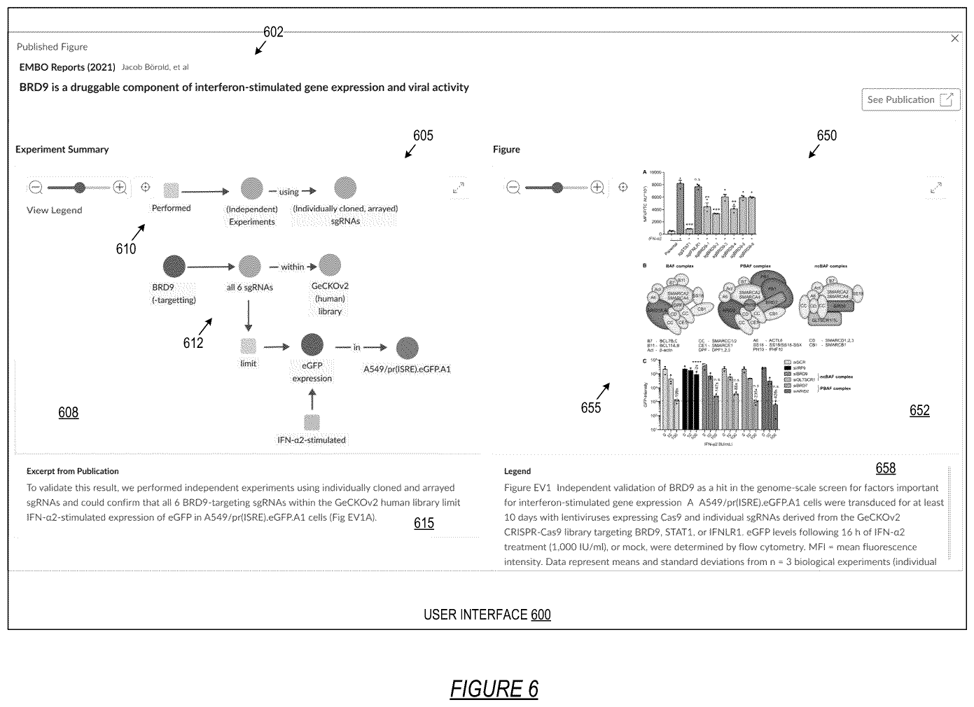

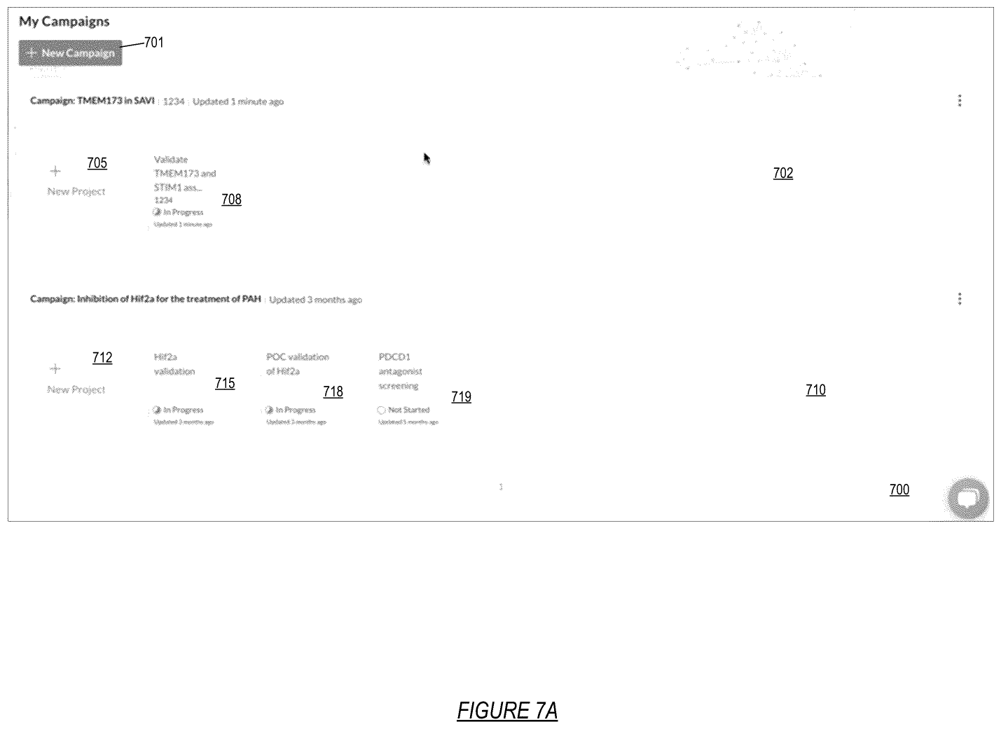

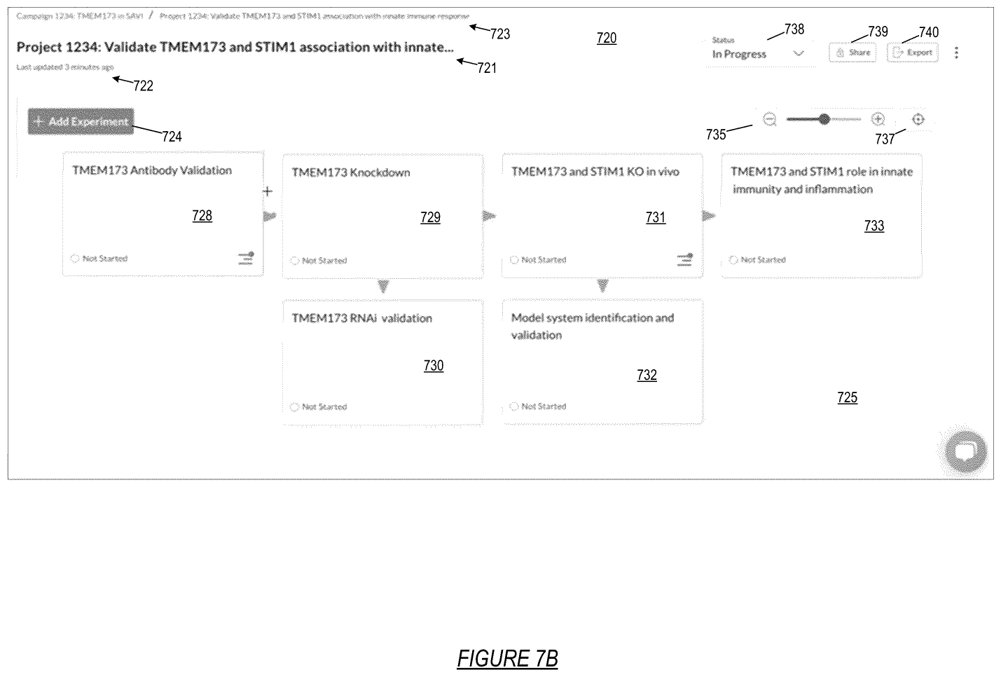

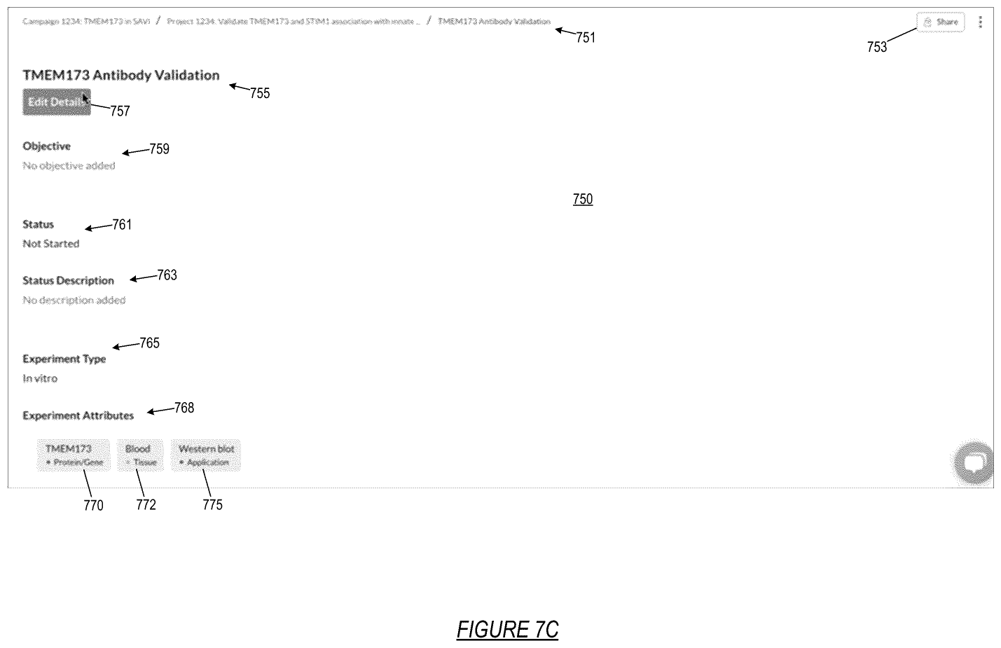

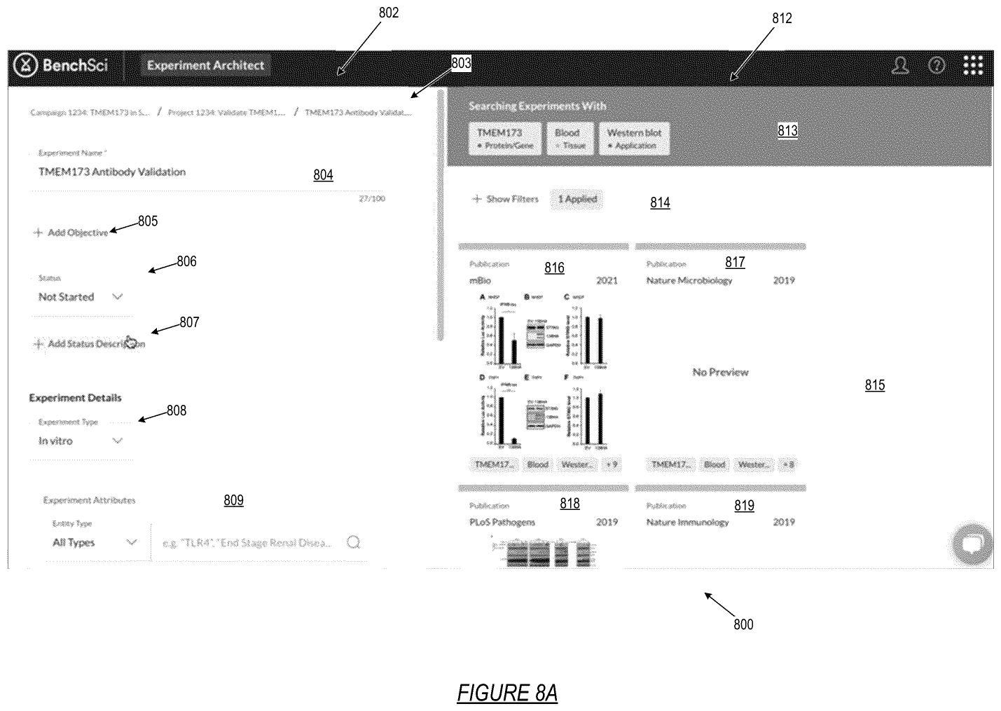

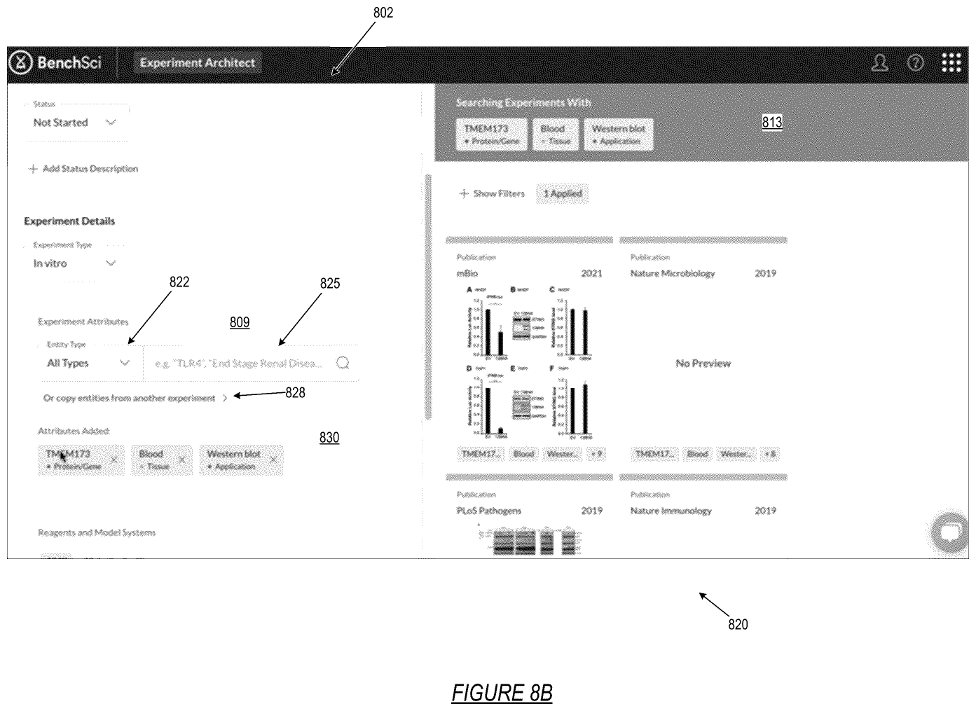

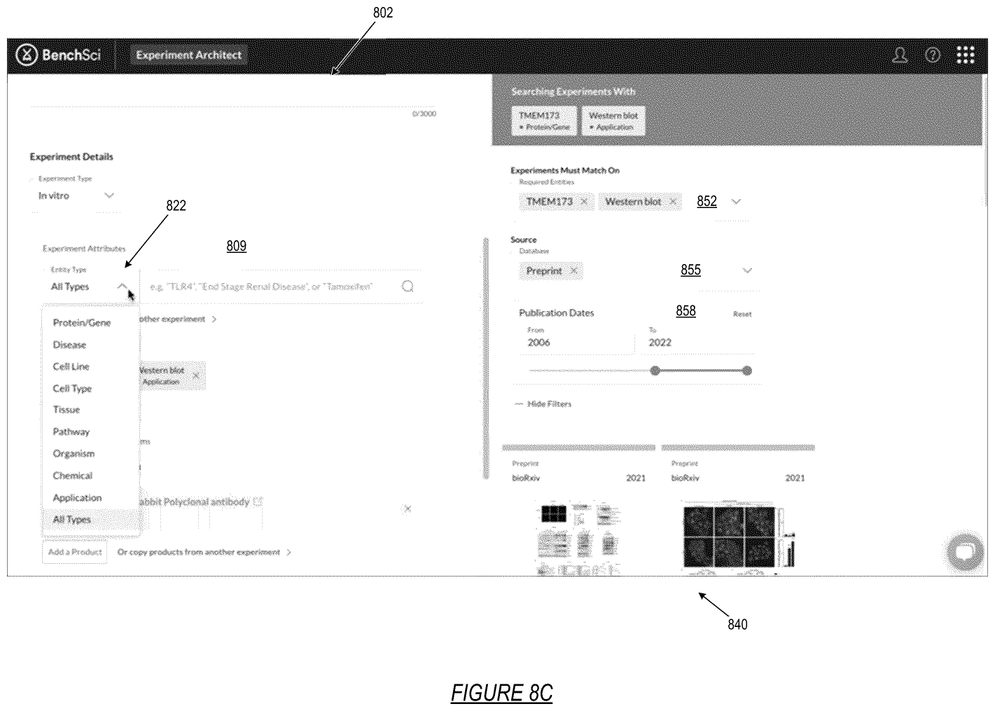

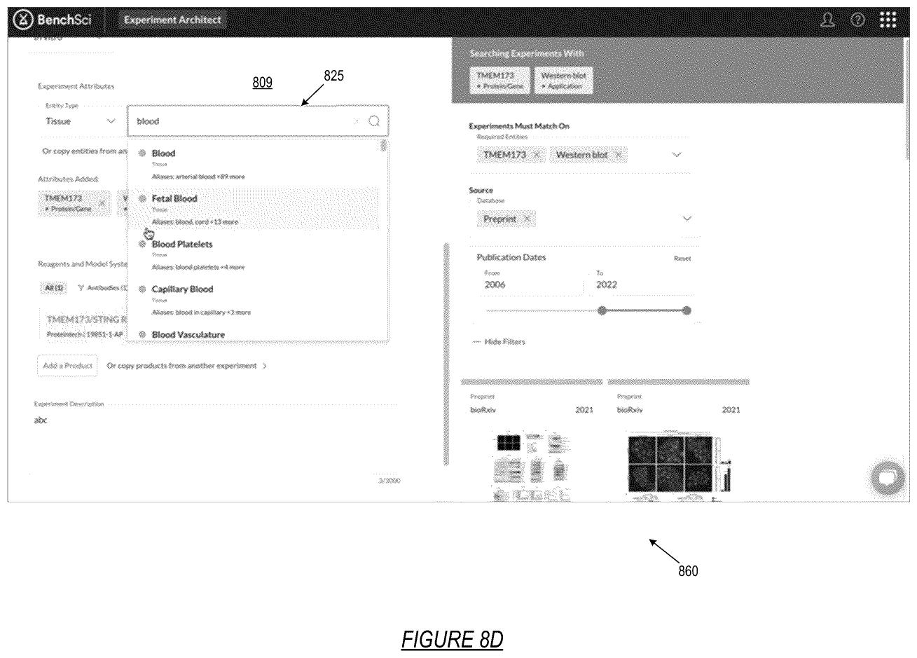

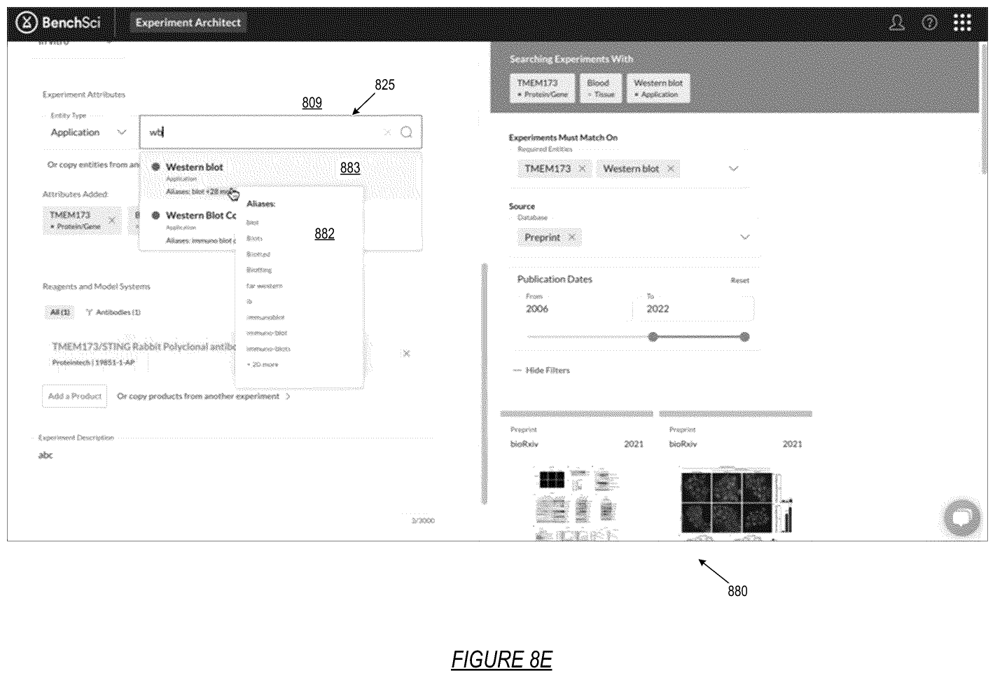

Turning to FIG. 3A, the file (302) is shown from which the sentence (305) is extracted, which is used to generate the tree (308), which is used to generate the graph (350) (of FIG. 3B). The file (302), the sentence (305), the tree (308), and the result graph (350) (of FIG. 3B) may be stored as electronic files, transmitted in messages, and displayed on a user interface.

The file (302) is a collection of biomedical information, which may include, but is not limited to, a writing of biomedical literature with sentences and figures stored as text and images. Different sources of biomedical information may be used. The file (302) is processed to extract the sentence (305).

The sentence (305) is a sentence from the file (302). The sentence (305) is stored as a string of characters. In one embodiment, the sentence (305) is tokenized to identify the locations of entities within the sentence (305). For example, the entities recognized from the sentence (305) may include “CCN2”, “LRP6”, “HCC”, and “HCC cell lines”. The sentence (305) is processed to generate the tree (308).

The tree (308) is a data structure that identifies semantic relationships of the words of the sentence (305). The tree (308) includes the leaf nodes (312), the intermediate nodes (315), and the root node (318).

The leaf nodes (312) correspond to the words from the sentence (305). The leaf nodes have no child nodes. The leaf nodes have parent nodes in the intermediate nodes (315).

The intermediate nodes (315) include values that identify the parts of speech of the leaf nodes (312). The intermediate nodes (315) having leaf nodes as direct children nodes identify the parts of speech of the words represented by the leaf nodes. The intermediate nodes (315) that do not have leaf nodes as direct children nodes identify the parts of speech of groups of one or more words, i.e., phrases, of the sentence (305).

The root node (318) is the top of the tree (308). The root node (318) has no parent node.

Turning to FIG. 3B, the result graph (350) is a data structure that represents the sentence (305) (of FIG. 3A). The result graph (350) may be generated from the sentence (305) and the tree (308). The nodes of the result graph (350) represent nouns (e.g., “CCN2”, “HCC”, etc.) and verbs (e.g., “up-regulated”, “are”, etc.) from the sentence (305). The edges (355) identify semantic relationships (e.g., subject “sub”, verb “vb”, adjective “adj”) between the words of the nodes (352) of the sentence (305) (of FIG. 3A). The result graph (350) is a directed acyclic graph.

Turning to FIG. 4, the image (402) is shown from which the structured text (405) is generated, which is used to generate the result graph (408). The image (402), the structured text (405), and the result graph (408) may be stored as electronic files, transmitted in messages, and displayed on a user interface.

The image (402) is a figure from a file (e.g., the file (302) of FIG. 3A, which may be from a biomedical publication). In one embodiment, the image (402) is an image file that is included with or as part of the file (302) of FIG. 3A. In one embodiment, the image (402) is extracted from an image of a page of a publication stored as the file (302) of FIG. 3A. The image (402) includes three panels labeled “A”, “B”, and “C”. The “B” panel includes three subpanels labeled “BAF complex”, “PBAF complex”, and “ncBAF complex”. The image (402) is processed to recognize the locations of the panels, subpanels, and text using machine learning models. After being located, the text from the image is recognized and stored as text (i.e., strings of characters). The panel, subpanel, and text locations along with the recognized text are processed to generate the structured text (405).

The structured text (405) is a string of text characters that represents the image (402). In one embodiment, the structured text (405) includes nested lists that form a hierarchical structure patterned after the hierarchical structure of the panels, subpanels, and text from the image (402). The structured text (405) is processed to generate the result graph (408).

The result graph (408) is a data structure that represents the figure, corresponding to the image (402), from a file (e.g., the file (302) of FIG. 3A). The result graph (408) includes nodes and edges. The nodes represent nouns and verbs identified in the structured text (405). The edges may represent the nested relationships between the panels, subpanels, and text of the image (402) described in the structured text (405).

Turning to FIG. 5, the tagged sentence (502) is generated from a sentence and used to generate the updated result graph (505). The tagged sentence (502) and the updated result graph (505) may be stored as electronic files, transmitted in messages, and displayed on a user interface.

The tagged sentence (502) is a sentence from a file that has been processed to generate the updated result graph (505). The sentence from which the tagged sentence is derived is input to a model to tag the entities in the sentence to generate the tagged sentence (502). The model may be a rules-based model, an artificial intelligence model, combinations thereof, etc.

As an example, the underlined portion (“INSR and PIK3R1 levels were not altered in TNF-alpha treated myotubes”) is tagged by the model. The terms “INSR”, “PIK3R1”, and “TNF-alpha” may be tagged as one type of entity that is presented as green when displayed on a user interface. The term “not” is tagged and may be displayed as orange. The terms “altered” and “treated” are tagged and may be displayed as pink. The term “myotubes” is tagged and may be displayed as red. After being identified in the sentence, the tags may be applied to the graph to generate the updated result graph (505).

The updated result graph (505) is an updated version of a graph of the sentence used to generate the tagged sentence (502). The graph is updated to label the nodes of the graph with the tags from the tagged sentence. For example, the nodes corresponding to “INSR” and “PIK3R1” are labeled with tags identified in the tagged sentence and may be displayed as green. The node corresponding to “altered” is tagged and displayed as pink. The node corresponding to “myotubes” is tagged and displayed as red.

Turning to FIG. 6, the user interface (600) displays information from a file, which may be a publication of biomedical literature. Different sources of files may be used. The user interface (600) may display the information on a user device after receiving a response to a request for the information transmitted to a server application. For example, the request may be for a publication that includes evidence linking the proteins “BRD9” and “A549”. The user interface displays the header section (602), the summary section (605), and the figure section (650).

The header section (602) includes text identifying the file being displayed. In one embodiment, the text in the header section (602) includes the name of the publication, the name of the author, the title of the publication, etc., which may be extracted from the file. Additional sources of information may be used, including patents, ELN data, summary documents, portfolio documents, scientific data in raw/table form, presentations, etc., and similar information may be extracted.

The summary section (605) displays information from the text of the file identified in the header section (602). The summary section (605) includes the graph section (608) and the excerpt section (615).

The graph section (608) includes the result graphs (610) and (612). The result graphs (610) and (612) were generated from the sentence displayed in the excerpt section (615). The result graph (612) shows the link between the proteins “BRD9” and “A549”, which conforms to the request that prompted the response with the information displayed in the user interface (600).

The excerpt section (615) displays a sentence from the file identified in the header section (602). The sentence in the excerpt section (615) is the basis from which the result graphs (610) and (612) were generated by tokenizing the sentence, generating a tree from the tokens, and generating the result graphs (610) and (612) from the tokens and tree.

The figure section (650) displays information from the figures of the file identified in the header section (602). The figure section (650) includes the image section (652) and the legend section (658).

The image section (652) displays the image (655). The image (655) was extracted from the file identified in the header section (602). The image (655) corresponds to the text from the legend section (658). The image (655) corresponds to the result graph (612) because the sentence shown in the excerpt section (615) identifies the figure (“Fig EV1A”) that corresponds to the image (655).

The legend section (658) displays the text of the legend that corresponds to the figure of the image (655). In one embodiment, the text of the legend section (655) may be processed to generate one or more graphs from the sentence in the legend section (658).

FIGS. 7A through 7H, 8A through 8D, 9A through 9C, 10A through 10B, and 11A through 11G illustrate user interfaces that display evidence graphs and record data for experiment records. In one embodiment, the data in the user interfaces may be generated by a server and displayed on a user device.

Turning to FIG. 7A, the user interface (700) displays records for campaigns, projects, and experiments with user interface elements. A record of a campaign may include the records for multiple projects. A record of a project may include multiple experiment records. The user interface includes the new campaign element (701) and the campaign elements (702) and (710).

The new campaign element (701) is a user interface for new campaigns. Selecting the new campaign element (701) brings up a window with additional user interface elements that a user may interact with to generate a new campaign.

The campaign element (702) is a user interface element that displays information about a campaign and corresponding projects. The campaign element (702) includes the title “TMEM173 in SVI” and a last updated value of “updated 1 minute ago”. The title may be selected by the user when creating the campaign. The last updated value is determined by the system and identifies when the most recent change to a record corresponding to the campaign, including the campaign record, project records, experiment records, etc. The campaign element (700) includes the new project element (705) and the project element (708).

The new project element (705) is a user interface element for creating new projects in the campaign corresponding to the campaign element (702). Selecting the new project element (705) brings up a window with additional user interface elements that a user may interact with to generate a new project for the campaign corresponding to the campaign element (702).

The project element (708) is a user interface element that displays information about a project. The project element (708) displays at least a portion of the title of the project (“Validate TMEM173 and STIM1 ass . . . ”), a progress indicator, and a last updated value. The progress indicator includes an icon and text to identify the progress of the project (“In Progress”). The last updated value indicates the records of the project were last updated about “1 minute ago”.

The campaign element (710) includes the project elements (712), (715), (718), and (719). The campaign element (710) and the project elements (712), (715), (718), and (719) perform similar functions and include similar information as that described with respect to the campaign element (702), the new project element (705), and the project element (708) for different campaigns and projects.

Turning to FIG. 7B, the user interface (720) displays user interfaces elements with information of a project and experiments of a campaign. The user interface (720) is displayed after selecting the project element (708) (of FIG. 7A) from the user interface (700) (of FIG. 7A). The user interface (720) displays the title (721) of the project, an update status (722) of the project, and a path (723) of the project. The path (723) of the project displays the title of the campaign with the title of the project. The user interface (720) includes the project view (725), the status element (738), the share element (739), and the export element (740).

The project view (725) is a user interface element that displays a view of a project recorded by the system. The project view (725) shows a flow chart with the experiment cards (728), (729), (730), (731), (732), and (733). The experiment cards (728) through (733) may be rearranged by dragging the experiment cards (728) through (733) around within the project view (725). The project view (725) further includes the add experiment element (724), the zoom element (735) and the recenter element (737).

The experiment cards (728) through (733) are user interface elements that display information about the records of experiments that correspond to the project of the project view (725). Each of the experiment cards (728) through (733) include interface elements to display titles of the experiments represented by the experiment cards (728) through (733) as well as the status of the experiments represented by the experiment cards (728) through (733).

The add experiment element (724), of the project view (725), is a user interface element for adding a new experiment, shown as a new experiment card, to the project corresponding to the project view (725). Selecting the add experiment element (724) may open an experiment editor view, which is further described with the FIGS. 8A through 12.

The zoom element (735) is a user interface element to adjust a zoom of the project view (725). Zooming in shows fewer experiments and zooming out shows more experiments.

The recenter element (737) adjusts the zoom and panning of the project view (725). The zoom and panning may be adjusted to show all of the experiment cards (728) through (733) within the project view (725).

The status element (738) is a user interface element that records and identifies the status of the project corresponding to the project view (725). In one embodiment, the status element (738) is a dropdown box from which a status identifier for the project may be selected. The status identifier selected for the project is “In Progress”. Additional status identifiers may include “Not Started”, “Completed”, etc.

The share element (739) is a user interface element that shares the project displayed in the user interface (720). Selecting the share element (739) may bring up a dialog box with text input boxes for e-mail addresses of the recipients. The dialog box may further include a link (e.g., a uniform resource locator (URL)) that, when accessed, displays the user interface (720) with the project view (725).

The export element (740) is a user interface element that may export information about the project displayed in the user interface (720). Selecting the export element (740) may bring up a dialog with multiple options for exporting project displayed in the user interface (720). In one embodiment, the display of the project view (725) may be converted to an image that is shared along with a link to the project. In one embodiment, your project may be exported by converting the information from the project view (725) and the experiment cards (728) through (733) to electronic slides that may also include links to the project and experiments.

Turning to FIG. 7C, the user interface (750) displays information from a record of an experiment. The user interface (750) is displayed in response to selection of the experiment card (728) (of FIG. 7B). The user interface (750) includes the path (751), the share element (753), the title (755), the edit element (757), the objective element (759), the status element (761), the status description element (763), the experiment type element (765), and the attribute list element (768).

The path (751) is a user interface element that displays information about the campaign, project, and experiment. The path (751) includes the title of the campaign, the title of the project, and the title of the experiment.

The share element (753) is a user interface element for sharing the record of the experiment displayed in the user interface (750). In one embodiment, selection of the shared element (753) brings up a dialog box (not shown) that may receive email addresses and provide a link that, when selected, loads the user interface (750) into the application that was used to select the link. The link may be sent to the e mail addresses identified in the dialog box.

The title (755) is a user interface element of the user interface (750). The title (755) identifies the title of the experiment displayed in the user interface (750).

The edit element (757) is a user interface element of the user interface (750). Selection of the edit element (757) may open an experiment editor view, which is further described with FIGS. 8A through 12.

The objective element (759) is a user interface element of the user interface (750). The objective element (759) displays the objective of the experiment displayed in the user interface (750). The objective may be stored as a text string.

The status element (761) is a user interface element of the user interface (750). The status element (761) displays the status of the experiment displayed in the user interface (750). The status may be a categorical value, which may be stored as a text string, a numerical value, etc.

The status description element (763) is a user interface element of the user interface (750). The status description element (763) displays the description of the status of the experiment displayed in the user interface (750). The description of the status may be stored as a text string.

The experiment type element (765) is a user interface element of the user interface (750). The experiment type element (765) displays the type of the experiment displayed in the user interface (750). The type may be a categorical value, which may be stored as a text string, a numerical value, etc.

The attribute list element (768) is a user interface element that identifies attributes used to search for biomedical information that is relevant to the experiment displayed in the user interface (750). The attributes identified in the attribute list element (768) may be selected by the user and may be identified by the system in response to processing other information about an experiment. For example, the title of the experiment may be processed to identify words from the title that match to words, phrases, or aliases of biomedical entities. The matches may be presented to the user and be selected as attributes for the experiment. The attribute list element (768) includes the attribute elements (770), (772), and (775).

The attribute elements (770), (772), and (775) identify the attributes associated with the experiment shown in the user interface (750). For example, the attribute element (770) includes a name and a type. The name identifies the name of the entity (defined in the ontology library) of the attribute and the type identifies the type of the attribute. The attribute element (770) is for an entity named “TMEM173”, which has the type “Protein/Gene”.

Turning to FIG. 8A, the user interface (800) interactively displays information that may be edited about an experiment. The user interface (800) includes the detail view (802) and the search view (812).

The detail view (802) is a user interface element that includes multiple additional user interface elements to edit the information about an experiment and stored to an experiment record. The detail view (802) includes the path (803), the title element (804), the objective element (805), the status element (806), the status description element (807), the experiment type element (808), and the attributes element (809).

The path (803) is a user interface element that displays information about the campaign, project, and experiment. The path (803) includes the title of the campaign, the title of the project, and the title of the experiment.

The title element (804) is a user interface element of the user interface (800). The title element (804) displays the title (also referred to as a “name”) of the experiment, which may be edited by the user.

The objective element (805) is a user interface element of the user interface (800). Selection of the objective element (805) may display a text input box that may receive a text string for the objective of the experiment.

The status element (806) is a user interface element of the user interface (800). The status element (806) is a dropdown box that may display a list of status identifiers for the experiment. The status identifier presently selected is “Not Started”.

The status description element (807) is a user interface element of the user interface (800). Selection of the status description element (807) may display a text input box that may receive a text string for the description of the status of the experiment.

The experiment type element (808) is a user interface element of the user interface (800). The experiment type element (808) is a dropdown box that may display a list of types for the experiment. The type presently selected is “In Vitro”.

The attributes element (809) is a user interface element of the user interface (800). The attributes element (809) is further described with FIG. 8B.

The search view (812) is a user interface element that includes multiple additional user interface elements to search for information related to the experiment shown in the detail view (802). The search view (812) includes the attribute list element (813), the filter element (814), and the results element (815).

The attribute list element (813) is a user interface element of the search view (812). The attribute list element (813) lists the attributes that have been identified in the attributes element (809).

The filter element (814) is a user interface element of the search view (812). The filter element (814) is used to filter the attributes and to filter the results displayed in the results element (815). The filter element (814) is further described in FIGS. 9A through 9C.

The results element (815) is a user interface element of the search view (812). The results element (815) display the results of searching the result graphs generated by the system for the attributes identified in the attribute list element (813) and filtered with the filter element (814). The results element (815) includes the result cards (816), (817), (818), and (819).

The result cards (816) through (819) display evidence from sources of biomedical information that have been analyzed by the system. For example, the result card (816) includes an image from a publication that is evidence of a result of an experiment. The result card further includes a type identifier (“Publication”), the title of the source (a publication named “mBio”), the date of the source (“2021”), and a list of attributes that the system has identified for entities present the in the source. For example, the publication of the result card (816) includes a reference to the “TMEM173” protein/gene. The results element (815) is further described in FIG. 10A.

Turning to FIG. 8B, the user interface (820) is updated from the user interface (800) of FIG. 8A. The user interface (820) is updated by scrolling down in the detail view (802) to reveal more of the attributes element (809). The attributes element (809) includes the type element (822), the name element (825), the copy element (828), and the list element (830).

The type element (822) is a user interface element within the attributes element (809). The type element (822) is used to select a type of an attribute. The type element (822) is further described in FIG. 8C.

The name element (825) is a user interface element within the attributes element (809). The name element (825) is used to select a name of an attribute, which matches to an entity of the ontology library. The name element (825) is further described with FIGS. 8D and 8E.

The copy element (828) is a user interface element within the attributes element (809). In one embodiment, selection of the copy element (828) brings up a dialog box from which the user may select another experiment stored on the system. The attributes from the other experiment may then be imported into the present experiment and displayed in the list element (830).

The list element (830) is a user interface element within the attributes element (809). The list element (830) displays and interactive list of the attributes that have been identified to correspond with the experiment displayed in the user interface (820). Each attribute is displayed in a user interface element that includes the name of the attribute (e.g., “TMEM173”, “Blood”, “Western blot”, etc.) and the type of entity of the attribute (e.g., “Protein/Gene”, “Tissue”, “Application”, etc.). The user interface element of an attribute additionally includes and “X” icon that, when selected, removes the attribute from the experiment. Adding a new attribute to the attributes element (809) may trigger the display of the new attribute in the attribute list element (813).

Turning to FIG. 8C, the user interface (840) is updated from the user interface (820) of FIG. 8B. The user interface (840) is updated to show the selection of the type element (822). Upon selection, the type element (822) displays a dropdown box that includes items for the different types of entities recognized by the system and defined in the ontology library. The types of entities include proteins, genes, diseases, cell lines, cell types, tissues, pathways, organisms, chemicals, applications, etc.

Additionally, the user interface (840) is updated in response to selection of the filter element (814) (of FIG. 8A). The user interface (840) to show the attribute filter (852) (described with FIG. 9A), the source filter (855) (described with FIG. 9B), and the date filter (858) (described with FIG. 9C).

Turning to FIG. 8D, the user interface (860) is updated to show the selection of the name element (825). After selecting the name element (825), the user may enter text into the name element (825). The text is compared to the names and aliases of entities defined in the ontology library. The user may select the name of the entity for the attribute to be added to the experiment.

Turning to FIG. 8E, the user interface (880) is updated to show the popup menu (882). After selecting to view the aliases of an item in the list of entity names (883), the popup menu (882) is displayed to show the name aliases that correspond to the name of the entity in the list of entity names (883).

Turning to FIG. 9A, the user interface (900) is updated after selection of the attribute filter (852). Selection of the attribute filter (852) brings up the dropdown box element (902). With the dropdown box element (902), the user may select which attributes are used to search for sources (e.g., publications) of biomedical information that are related to the experiment. After changing which attributes are used to search the biomedical information, the results element (915) is updated from the results element (815) (of FIG. 8A) to show evidence from sources of biomedical information that are relevant to the experiment.

Turning to FIG. 9B, the user interface (930) is updated after selection of the source filter (855). Selection of the source filter (855) brings up the dropdown box element (932). With the dropdown box element (932), the user may select a category of sources to which to restrict the searching of biomedical information. The categories include “Publication”, “Internal”, “Preprint”, etc. Sources that are “publications” have been published and include journal articles. Sources that are “Internal” include papers written within a company that are not shared outside the company. Sources that are “Preprint” include papers that have been published but not peer reviewed. After selecting the types of sources that may be used, the results element (915) may be updated.

Turning to FIG. 9C, the user interface (960) is updated after selection of the date filter (858). The date filter (858) includes text boxes and a slider to set the earliest publication date and latest publication date. After interaction with the date filter (858), the results in the results element (915) are updated to show sources with dates of publication that are after the earliest publication date and prior to the latest publication date.

Turning to FIG. 10A, the user interface (1000) is updated from the user interface (800) (of FIG. 8A). The user interface (1000) is updated to scroll down in the search view (812) to show results in the results element (815).

Additionally, the result card (818) is updated. The result card (818) is updated responsive to the location of the mouse pointer hovering over the location of the result card (818). Upon hovering, the image from the result card is replaced with text that corresponds to the image. The text includes a source type identifier (“Publication”), a name of the source (“PLoS Pathogens”), a date of the source (“2019”), the authors of the source (“Liya Ye, Qiang Zhang, Tianzi Liuyu, et al.”), and a sentence that is related to the image. The sentence related to the image may describe the image and be derived from the result graph, for the image, that was generated from the source.

Turning to FIG. 10B, the user interface (1020) is displayed. The user interface (1020) is displayed in response to the user selecting one of the result cards from the results element (815) (of FIGS. 10A and 8A) Similar to the user interface (600) (of FIG. 6), the user interface (1020) displays the summary section (1022), and the figure section (1025). The summary section (1022) includes the result graph (1023) and the figure section includes the image (1026). The result graph (1026) is generated from the source material (e.g., a publication of biomedical information) and describes entities, and relations between the entities, from the image (1026).

Turning to FIG. 10C, the user interface (1050) is displayed. The user interface (1050) is updated from the user interface (1020) by scrolling down. The user interface (1050) displays attributes that may be selected for an experiment (e.g., the experiment corresponding to the user interface (800) of FIG. 8A). The attributes displayed in the user interface (1050) correspond to entities that were identified in the source of biomedical information from which the image (1026) was extracted and the result graph (1023) was generated.

Turning to FIG. 10D, the user interface (1060) is displayed. The user interface (1060) is scrolled down further to display additional attributes that may be selected for an experiment. The attributes are aggregated by the type of attribute. For example, the attributes “Thp-1 Cell” and “U-937 Cell” are aggregated to the “Cell Line” type of attribute. The user interface element for the attribute “Blood” includes a check mark to indicate that the attribute “Blood” is already part of the experiment. Other user interface elements, including the user interface element for “Plural Effusion” includes a plus sign to indicate that selection of these are emphasis element with a plus sign will add the attribute to the experiment. Each user interface element for an attribute includes the name of the attribute and the type of the attribute.

Turning to FIG. 11, the user interface (1100) is updated to scroll down in the detail view (802). The user interface (1100) shows the product interface (1102).

The product interface (1102) interactively displays the products that are used with the experiment. For the experiment being edited with the user interface (1100), two products are included and shown with the product element (1105) and the product element (1108). The product elements identify the name of the entity as defined in the biomedical information of the ontology library, the manufacturer of the product, and an identification number (e.g., an SKU number) from the manufacturer that identifies the product. For example, the product element (1105) is for the entity named “TMEM173/STING Rabbit Polyclonal antibody” from the manufacturer “Proteintech” with a manufacturer identification number of “19851 1-AP”.

Turning to FIG. 12, the user interface (1200) is updated to scroll down in the detail view (802). The user interface (1200) shows the protocol element (1202). Selecting the protocol element (1202) may bring up a dialog box, which may receive and address for a link to a document that describes the protocol to use for the experiment.

Embodiments of the invention may be implemented on a computing system. Any combination of a mobile, a desktop, a server, a router, a switch, an embedded device, or other types of hardware may be used. For example, as shown in FIG. 13A, the computing system (1300) may include one or more computer processor(s) (1302), non-persistent storage (1304) (e.g., volatile memory, such as a random access memory (RAM), cache memory), persistent storage (1306) (e.g., a hard disk, an optical drive such as a compact disk (CD) drive or a digital versatile disk (DVD) drive, a flash memory, etc.), a communication interface (1312) (e.g., Bluetooth interface, infrared interface, network interface, optical interface, etc.), and numerous other elements and functionalities.

The computer processor(s) (1302) may be an integrated circuit for processing instructions. For example, the computer processor(s) (1302) may be one or more cores or micro-cores of a processor. The computing system (1300) may also include one or more input device(s) (1310), such as a touchscreen, a keyboard, a mouse, a microphone, a touchpad, an electronic pen, or any other type of input device.

The communication interface (1312) may include an integrated circuit for connecting the computing system (1300) to a network (not shown) (e.g., a local area network (LAN), a wide area network (WAN) such as the Internet, a mobile network, or any other type of network) and/or to another device, such as another computing device.

Further, the computing system (1300) may include one or more output device(s) (1308), such as a screen (e.g., a liquid crystal display (LCD), a plasma display, a touchscreen, a cathode ray tube (CRT) monitor, a projector, or other display device), a printer, an external storage, or any other output device. One or more of the output device(s) (1308) may be the same or different from the input device(s) (1310). The input and output device(s) (1310 and (1308)) may be locally or remotely connected to the computer processor(s) (1302), non-persistent storage (1304), and persistent storage (1306). Many different types of computing systems exist, and the aforementioned input and output device(s) (1310 and (1308)) may take other forms.

Software instructions in the form of computer readable program code to perform embodiments of the invention may be stored, in whole or in part, temporarily or permanently, on a non-transitory computer readable medium such as a CD, a DVD, a storage device, a diskette, a tape, flash memory, physical memory, or any other computer readable storage medium. Specifically, the software instructions may correspond to computer readable program code that, when executed by a processor(s), is configured to perform one or more embodiments of the invention.

The computing system (1300) in FIG. 13A may be connected to or be a part of a network. For example, as shown in FIG. 13B, the network (1320) may include multiple nodes (e.g., node X (1322), node Y (1324)). Each node may correspond to a computing system, such as the computing system (1300) shown in FIG. 13A, or a group of nodes combined may correspond to the computing system (1300) shown in FIG. 13A. By way of an example, embodiments of the invention may be implemented on a node of a distributed system that is connected to other nodes. By way of another example, embodiments of the invention may be implemented on a distributed computing system having multiple nodes, where each portion of the invention may be located on a different node within the distributed computing system. Further, one or more elements of the aforementioned computing system (1300) may be located at a remote location and connected to the other elements over a network.

Although not shown in FIG. 13B, the node may correspond to a blade in a server chassis that is connected to other nodes via a backplane. By way of another example, the node may correspond to a server in a data center. By way of another example, the node may correspond to a computer processor or micro-core of a computer processor with shared memory and/or resources.

The nodes (e.g., node X (1322), node Y (1324)) in the network (1320) may be configured to provide services for a client device (1326). For example, the nodes may be part of a cloud computing system. The nodes may include functionality to receive requests from the client device (1326) and transmit responses to the client device (1326). The client device (1326) may be a computing system, such as the computing system (1300) shown in FIG. 13A. Further, the client device (1326) may include and/or perform all or a portion of one or more embodiments of the invention.

The computing system (1300) or group of computing systems described in FIGS. 13A and 13B may include functionality to perform a variety of operations disclosed herein. For example, the computing system(s) may perform communication between processes on the same or different system. A variety of mechanisms, employing some form of active or passive communication, may facilitate the exchange of data between processes on the same device. Examples representative of these inter-process communications include, but are not limited to, the implementation of a file, a signal, a socket, a message queue, a pipeline, a semaphore, shared memory, message passing, and a memory-mapped file. Further details pertaining to a couple of these non-limiting examples are provided below.

Based on the client-server networking model, sockets may serve as interfaces or communication channel end-points enabling bidirectional data transfer between processes on the same device. Foremost, following the client-server networking model, a server process (e.g., a process that provides data) may create a first socket object. Next, the server process binds the first socket object, thereby associating the first socket object with a unique name and/or address. After creating and binding the first socket object, the server process then waits and listens for incoming connection requests from one or more client processes (e.g., processes that seek data). At this point, when a client process wishes to obtain data from a server process, the client process starts by creating a second socket object. The client process then proceeds to generate a connection request that includes at least the second socket object and the unique name and/or address associated with the first socket object. The client process then transmits the connection request to the server process. Depending on availability, the server process may accept the connection request, establishing a communication channel with the client process, or the server process, busy in handling other operations, may queue the connection request in a buffer until server process is ready. An established connection informs the client process that communications may commence. In response, the client process may generate a data request specifying the data that the client process wishes to obtain. The data request is subsequently transmitted to the server process. Upon receiving the data request, the server process analyzes the request and gathers the requested data. Finally, the server process then generates a reply including at least the requested data and transmits the reply to the client process. The data may be transferred, more commonly, as datagrams or a stream of characters (e.g., bytes).

Shared memory refers to the allocation of virtual memory space in order to substantiate a mechanism for which data may be communicated and/or accessed by multiple processes. In implementing shared memory, an initializing process first creates a shareable segment in persistent or non-persistent storage. Post creation, the initializing process then mounts the shareable segment, subsequently mapping the shareable segment into the address space associated with the initializing process. Following the mounting, the initializing process proceeds to identify and grant access permission to one or more authorized processes that may also write and read data to and from the shareable segment. Changes made to the data in the shareable segment by one process may immediately affect other processes, which are also linked to the shareable segment. Further, when one of the authorized processes accesses the shareable segment, the shareable segment maps to the address space of that authorized process. Often, only one authorized process may mount the shareable segment, other than the initializing process, at any given time.

Other techniques may be used to share data, such as the various data sharing techniques described in the present application, between processes without departing from the scope of the invention. The processes may be part of the same or different application and may execute on the same or different computing system.

Rather than or in addition to sharing data between processes, the computing system performing one or more embodiments of the invention may include functionality to receive data from a user. For example, in one or more embodiments, a user may submit data via a graphical user interface (GUI) on the user device. Data may be submitted via the graphical user interface by a user selecting one or more graphical user interface widgets or inserting text and other data into graphical user interface widgets using a touchpad, a keyboard, a mouse, or any other input device. In response to selecting a particular item, information regarding the particular item may be obtained from persistent or non-persistent storage by the computer processor. Upon selection of the item by the user, the contents of the obtained data regarding the particular item may be displayed on the user device in response to the user's selection.

By way of another example, a request to obtain data regarding the particular item may be sent to a server operatively connected to the user device through a network. For example, the user may select a uniform resource locator (URL) link within a web client of the user device, thereby initiating a Hypertext Transfer Protocol (HTTP) or other protocol request being sent to the network host associated with the URL. In response to the request, the server may extract the data regarding the particular selected item and send the data to the device that initiated the request. Once the user device has received the data regarding the particular item, the contents of the received data regarding the particular item may be displayed on the user device in response to the user's selection. Further to the above example, the data received from the server after selecting the URL link may provide a web page in Hyper Text Markup Language (HTML) that may be rendered by the web client and displayed on the user device.

Once data is obtained, such as by using techniques described above or from storage, the computing system, in performing one or more embodiments of the invention, may extract one or more data items from the obtained data. For example, the extraction may be performed as follows by the computing system (1300) in FIG. 13A. First, the organizing pattern (e.g., grammar, schema, layout) of the data is determined, which may be based on one or more of the following: position (e.g., bit or column position, Nth token in a data stream, etc.), attribute (where the attribute is associated with one or more values), or a hierarchical/tree structure (consisting of layers of nodes at different levels of detail-such as in nested packet headers or nested document sections). Then, the raw, unprocessed stream of data symbols is parsed, in the context of the organizing pattern, into a stream (or layered structure) of tokens (where each token may have an associated token “type”).

Next, extraction criteria are used to extract one or more data items from the token stream or structure, where the extraction criteria are processed according to the organizing pattern to extract one or more tokens (or nodes from a layered structure). For position-based data, the token(s) at the position(s) identified by the extraction criteria are extracted. For attribute/value-based data, the token(s) and/or node(s) associated with the attribute(s) satisfying the extraction criteria are extracted. For hierarchical/layered data, the token(s) associated with the node(s) matching the extraction criteria are extracted. The extraction criteria may be as simple as an identifier string or may be a query presented to a structured data repository (where the data repository may be organized according to a database schema or data format, such as XML).

The extracted data may be used for further processing by the computing system. For example, the computing system (1300) of FIG. 13A, while performing one or more embodiments of the invention, may perform data comparison. Data comparison may be used to compare two or more data values (e.g., A, B). For example, one or more embodiments may determine whether A>B, A=B, A=B, A<B, etc. The comparison may be performed by submitting A, B, and an opcode specifying an operation related to the comparison into an arithmetic logic unit (ALU) (i.e., circuitry that performs arithmetic and/or bitwise logical operations on the two data values). The ALU outputs the numerical result of the operation and/or one or more status flags related to the numerical result. For example, the status flags may indicate whether the numerical result is a positive number, a negative number, zero, etc. By selecting the proper opcode and then reading the numerical results and/or status flags, the comparison may be executed. For example, in order to determine if A>B, B may be subtracted from A (i.e., A−B), and the status flags may be read to determine if the result is positive (i.e., if A>B, then A−B>0). In one or more embodiments, B may be considered a threshold, and A is deemed to satisfy the threshold if A=B or if A>B, as determined using the ALU. In one or more embodiments of the invention, A and B may be vectors, and comparing A with B requires comparing the first element of vector A with the first element of vector B, the second element of vector A with the second element of vector B, etc. In one or more embodiments, if A and B are strings, the binary values of the strings may be compared.

The computing system (1300) in FIG. 13A may implement and/or be connected to a data repository. For example, one type of data repository is a database. A database is a collection of information configured for ease of data retrieval, modification, re-organization, and deletion. A Database Management System (DBMS) is a software application that provides an interface for users to define, create, query, update, or administer databases.

The user, or software application, may submit a statement or query into the DBMS. Then the DBMS interprets the statement. The statement may be a select statement to request information, update statement, create statement, delete statement, etc. Moreover, the statement may include parameters that specify data, or data container (database, table, record, column, view, etc.), identifier(s), conditions (comparison operators), functions (e.g., join, full join, count, average, etc.), sort (e.g., ascending, descending), or others. The DBMS may execute the statement. For example, the DBMS may access a memory buffer, a reference or index a file for read, write, deletion, or any combination thereof, for responding to the statement. The DBMS may load the data from persistent or non-persistent storage and perform computations to respond to the query. The DBMS may return the result(s) to the user or software application.

The computing system (1300) of FIG. 13A may include functionality to present raw and/or processed data, such as results of comparisons and other processing. For example, presenting data may be accomplished through various presenting methods. Specifically, data may be presented through a user interface provided by a computing device. The user interface may include a GUI that displays information on a display device, such as a computer monitor or a touchscreen on a handheld computer device. The GUI may include various GUI widgets that organize what data is shown as well as how data is presented to a user. Furthermore, the GUI may present data directly to the user, e.g., data presented as actual data values through text, or rendered by the computing device into a visual representation of the data, such as through visualizing a data model.

For example, a GUI may first obtain a notification from a software application requesting that a particular data object be presented within the GUI. Next, the GUI may determine a data object type associated with the particular data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the particular data object and render a visual representation of the data values within a display device according to the designated rules for that data object type.

Data may also be presented through various audio methods. In particular, data may be rendered into an audio format and presented as sound through one or more speakers operably connected to a computing device.

Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

The above description of functions presents only a few examples of functions performed by the computing system (1300) of FIG. 13A and the nodes (e.g., node X (1322), node Y (1324)) and/or client device (1326) in FIG. 13B. Other functions may be performed using one or more embodiments of the invention.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method comprising:
receiving an entity identifier;
searching a plurality of result graphs using the entity identifier to identify a file comprising content corresponding to the entity identifier;
presenting a figure from the file in response to identifying the file,
wherein the plurality of result graphs comprises a result graph generated from the file and corresponding to the figure.

2. The method of claim 1, further comprising:
receiving the entity identifier in response to a user selection of the entity identifier.

3. The method of claim 1, further comprising:
receiving the entity identifier in response to processing a user selection to identify the entity identifier.

4. The method of claim 1, further comprising:
receiving the entity identifier in response to a user selection of an alias of an entity corresponding to the entity identifier.

5. The method of claim 1, further comprising:
receiving the entity identifier,
wherein the entity identifier corresponds to an entity node of the result graph and corresponds to an entity record of an ontology library, and
wherein the entity record defines an entity as one of a protein, a gene, a disease, a cell line, a cell type, a tissue, a pathway, an organism, a chemical, and an application.

6. The method of claim 1, further comprising:
searching the plurality of result graphs to identify an entity node, of the result graph, corresponding to the entity identifier.

7. The method of claim 1, further comprising:
presenting an evidence view adjacent to a results view;
receiving the entity identifier using the evidence view; and
presenting the figure in the results view.

8. The method of claim 1, further comprising:
processing one or more values of an experiment record to select the entity identifier; and
receiving the entity identifier in response to processing the one or more values of the experiment record.

9. The method of claim 1, further comprising:
identifying the file,
wherein the file comprises biomedical information, and
wherein the biomedical information comprises text and images.

10. The method of claim 1, further comprising:
presenting the figure, wherein the figure is evidence of biomedical information described in the file and by the result graph.

11. A system comprising:
an information controller configured to receive an entity identifier;
a search controller configured to search a plurality of result graphs; and
an application executing on one or more processors and configured for:
receiving, by the information controller, the entity identifier;
searching, by the search controller, the plurality of result graphs using the entity identifier to identify a file comprising content corresponding to the entity identifier;
presenting a figure from the file in response to identifying the file,
wherein the plurality of result graphs comprises a result graph generated from the file and corresponding to the figure.

12. The system of claim 11, wherein the application is further configured for:
receiving the entity identifier in response to a user selection of the entity identifier.

13. The system of claim 11, wherein the application is further configured for:
receiving the entity identifier in response to processing a user selection to identify the entity identifier.

14. The system of claim 11, wherein the application is further configured for:
receiving the entity identifier in response to a user selection of an alias of an entity corresponding to the entity identifier.

15. The system of claim 11, wherein the application is further configured for:
receiving the entity identifier,
wherein the entity identifier corresponds to an entity node of the result graph and corresponds to an entity record of an ontology library, and
wherein the entity record defines an entity as one of a protein, a gene, a disease, a cell line, a cell type, a tissue, a pathway, an organism, a chemical, and an application.

16. The system of claim 11, wherein the application is further configured for:
searching the plurality of result graphs to identify an entity node, of the result graph, corresponding to the entity identifier.

17. The system of claim 11, wherein the application is further configured for:
presenting an evidence view adjacent to a results view;
receiving the entity identifier using the evidence view; and
presenting the figure in the results view.

18. The system of claim 11, wherein the application is further configured for:
processing one or more values of an experiment record to select the entity identifier; and
receiving the entity identifier in response to processing the one or more values of the experiment record.

19. The system of claim 11, wherein the application is further configured for:
identifying the file,
wherein the file comprises biomedical information, and
wherein the biomedical information comprises text and images.

20. A non-transitory computer-readable medium storing program instructions that, when executed by one or more processors, cause a computing system to perform operations comprising:
receiving an entity identifier;
searching a plurality of result graphs using the entity identifier to identify a file comprising content corresponding to the entity identifier;
presenting a figure from the file in response to identifying the file,
wherein the plurality of result graphs comprises a result graph generated from the file and corresponding to the figure.
